(19) United States
(12) Patent Application Publication
(10) Pub. No.: US 2015/0273021 A1
(43) Pub. Date: Oct. 1, 2015

(54) COMPOSITIONS AND METHODS FOR SUSTAINED DELIVERY OF GLUCAGON-LIKE PEPTIDE (GLP-1) RECEPTOR AGONIST THERAPEUTICS

(71) Applicant: TUFTS UNIVERSITY, Medford,, MA (US)

(72) Inventors: David L. Kaplan, Concord, MA (US); Michael Lovett, Peabody, MA (US); Tuna Yucel, Medford, MA (US); Xiaoqin Wang, Winchester, MA (US)

(21) Appl. No.: 14/434,022

(22) PCT Filed: Oct. 11, 2013
(86) PCT No.: PCT/US13/64497

§ 371 Date: Apr. 7, 2015

Related U.S. Application Data
(60) Provisional application No. 61/712,590, filed on Oct. 11, 2012.

(51) Int. Cl. A61K 38/26; A61K 47/42; A61K 9/06; A61K 9/00

(52) U.S. Cl. CPC A61K 38/26; A61K 9/0024; A61K 47/42; A61K 9/06

(57) ABSTRACT

The present invention is directed to silk-based drug delivery compositions or compositions for sustained delivery of therapeutic agent(s), such as glucagon-like peptide (GLP-1) receptor agonists, as well as methods of making and using the same.

### CROSS REFERENCE TO RELATED APPLICATIONS

This application claims the benefit under 35 U.S.C. §119(e) of U.S. Provisional Application No. 61/712,590 filed Oct. 11, 2012, the content of which is incorporated herein by reference in its entirety.

### TECHNICAL FIELD

The present disclosure relates generally to silk-based drug delivery compositions for sustained delivery of molecules, such as therapeutic agent(s), as well as methods of using the same. In one aspect the present disclosure relates to silk-based drug-delivery compositions for sustained delivery of glucagon-like peptide (GLP-1) receptor agonists and methods for treatment of diabetes.

### BACKGROUND

Type 2 diabetes mellitus is the most common form of diabetes and is characterized by the inability of fat, liver, and muscle cells to recognize insulin or the inability to produce enough insulin. Due to this insulin resistance or deficiency, blood sugar does not enter into these cells, resulting in hyperglycemia. Millions of Americans have been diagnosed with Type 2 diabetes and it continues to grow as a public health burden.

Current therapeutic approaches focus on controlling blood glucose levels through a variety of mechanisms. These drugs include insulin sensitizers such as metformin (Glucophage, Glumetza), which decreases the amount of glucose absorbed from food and lowers glucose production in the liver, and pioglitazone (Actos), which makes tissues more sensitive to insulin. Other drugs are insulin secretagogues such as glyburide (DiaBeta, Glynase), glipizide (Glucotrol), glimepiride (Amaryl), repaglinide (Prandin), and nateglinide (Starlix), which stimulate the pancreas to produce more insulin, or sitagliptin (Januvia) and saxagliptin (Onglyza), which are dipeptidyl peptidase-4 (DPP-4) inhibitors that cause an increase in incretin levels (GLP-1) which inhibits glucagon release and stimulates insulin release. All of these drugs are prescribed as oral tablets, typically taken once per day. Alternatively, GLP-1 analogs such as exenatide (Byetta) and liraglutide (Victoza) are prescribed as daily subcutaneous injections. In addition, insulin therapy is also an option, ranging from rapid-acting to long-acting insulin as well as insulin pumps. These drugs include insulin lispro (Humalog) and insulin aspart (NovoLog), which are fast-acting, and insulin glargine (Lantus) and insulin detemir (Levemir), which are long-acting. These drugs are administered before or after meals (rapid-acting) or once daily as a subcutaneous injection (long-acting).

Because currently available therapeutics require daily administration, either orally or through subcutaneous injection, there exists a critical need for a sustained release formulation that can be administered once weekly, monthly, or longer. One such formulation is Bydureon, a once weekly version of exenatide developed by Amylin Pharmaceuticals, Eli Lilly & Co., and Alkermes, was recently approved by the Food and Drug Administration (FDA). This formulation is prepared using a complicated poly(D,L-lactide-co-glycolide) (PLGA) microsphere coacervation method and, due to the size of the particles, must be injected using a 23-gauge needle for subcutaneous administration. Moreover, poor pharmacokinetics requires higher dosage of exenatide as compared to the once daily Byetta (Kwak et al., Pharmaceutical Research, 2009, 26: 2504). These drugs comprise the majority of the market and offer the opportunity for a sustained drug delivery formulation such as silk fibroin to reduce the frequency of administration. This is particularly critical for therapies that require frequent subcutaneous injections.

Thus, there is a need for improved pharmaceutical compositions lacking potentially inflammatory degradation byproducts that provide sustained delivery of therapeutic agent(s) which are manufactured in a manner that minimizes the use of hazardous organic solvents.

### SUMMARY

One aspect of the present disclosure utilizes silk fibroin as the delivery system. Silk offers a wide array of advantages in comparison to more commonly used synthetic polymer systems such as PLGA. Silk fibroin is processed under all aqueous conditions and under ambient temperatures, as compared to organic solvents and high temperatures for PLGA, and the degradation byproducts of silk (amino acids) are non-inflammatory in comparison to the byproducts (acids) of PLGA. These features allow pharmaceuticals that are sensitive to changes in temperature, pH, and organic solvents such as proteins (e.g. antibodies), and peptides (e.g., exenatide, liraglutide) to be delivered using silk fibroin as the delivery vehicle without loss of activity. By processing these drug formulations under mild conditions, the structures of these molecules remain intact and long term drug efficacy is retained as compared to PLGA-based systems where the drug may lose activity due to processing using organic solvents or be degraded by acidic byproducts over time after administration.

Accordingly, in one aspect the present disclosure provides silk-based drug delivery compositions that provide sustained delivery of therapeutic agent(s). In addition to fostering patient compliance, such silk-based drug delivery composition exhibit excellent biocompatibility and non-inflammatory degradation products, such as peptides and amino acids. Therefore, use of silk in sustained release pharmaceutical formulations as a carrier can minimize immune response, and enhance stability of an active ingredient as compared to other polymeric formulations with acidic degradation byproducts (e.g., PLGA). Silk compositions can be processed in completely aqueous based solvents. Accordingly, such silk-based drug delivery compositions avoid the use of hazardous organic solvents that are used in the preparation of PLGA based sustained release formulations.

Generally, the silk-based drug delivery compositions described herein comprises a therapeutic agent dispersed or encapsulated in a silk fibroin matrix. The silk fibroin matrix can be in the form of silk fibroin hydrogels. Further, the hydrogels can be in the form of bulk gels or gel particles (micro-gels). Moreover, the silk-based drug delivery composition is capable of sustained delivery of the therapeutic agent in vivo.

In some embodiments, the silk-based drug-delivery composition described herein can further comprise a biocompatible polymer, such as polyethylene glycol (PEG).

In some embodiments, the silk-based drug-delivery composition described herein can further comprise albumin.

In another aspect, provided herein is a pharmaceutical composition. The pharmaceutical composition comprises a silk-based drug delivery composition described herein and a pharmaceutically acceptable excipient.

Provided herein are also kits comprising a silk-based drug delivery composition and instructions for use.

In yet another aspect, provided herein is a method for sustained delivery in vivo of a therapeutic agent. The method comprises administering a silk-based drug delivery composition described herein to a subject. For administering to a patient, the silk-based drug delivery composition can be formulated with a pharmaceutically acceptable excipient or carrier. The therapeutic agent can be delivered in a therapeutically effective amount over a period of time.

In still another aspect, provided herein is a method for treating diabetes in a subject. The method comprises administering a silk-based drug delivery composition described herein to a subject in need thereof. For treatment of diabetes, the therapeutic agent can be any agent known in the art for treatment of diabetes. In some embodiments, the therapeutic agent can be a GLP-1 receptor agonist. In some embodiments, GLP-1 receptor agonist includes exenatide or liraglutide. Advantageously, silk-based drug delivery compositions herein can be used to administered the therapeutic agent once every 1-6 months (e.g., once every 1-2 months, once every 3-6 months) instead of the usually more frequent administration (e.g., 1-3 times or more a week) of therapeutic agents for treatment of diabetes.

In some of the exemplary embodiments of the silk-based drug delivery composition described herein, GLP-1 receptor agonists exenatide and liraglutide were used as exemplary therapeutic agents. In the specific case of the GLP-1 receptor agonists, the exenatide-loaded silk hydrogels demonstrated sustained release at estimated therapeutic levels for 1 week in vivo and greater than 1 month in vitro. Improvement of release profile to 2-3 months at therapeutic level can be obtained using a high drug loading in the hydrogels. Without wishing to be bound by a theory, this formulation can result in a significant reduction in the number of injections for patients suffering from type 2 diabetes mellitus. The release kinetics of the formulations can be further adjusted such that patients only require an injection every 3-6 months, a marked improvement from the current dosing of once daily.

### BRIEF DESCRIPTION OF THE DRAWINGS

FIG. 1 is a line graph showing Liraglutide concentrations in vitro for select silk hydrogel formulations. Formulations have different silk (2%, 4%) concentrations with fixed liraglutide (0.42%) concentration (w/v). Legend: S: Silk, L: Liraglutide.

FIG. 2 is a line graph showing plasma Exenatide concentrations. 2% Active is group 1 (2% silk, 0.06% Exenatide), 4% Active is group 2 (4% silk, 0.06% Exenatide), and PosCtrl is group 5 (0.06% Exenatide solution).

FIG. 3 is a line graph showing Exenatide concentrations in vitro for select silk hydrogel formulations with promising release kinetics. Formulations have different silk (8%, 16%) and Exenatide (0.06%, 0.12%) concentrations (w/v). Target release rate is based on the current dosing regimen of 10 μg/day, and assuming a 1 mL injection of silk hydrogel formulation. Legend: S: Silk, E: Exenatide.

FIG. 4 is a line graph showing Exenatide concentrations in vitro for silk hydrogel formulations with and without PEG or PEO. Formulations have equal silk (10%) and exenatide (0.06%) concentrations (w/v), with variations in PEG (MW 10,000, 0.25%, 1%, and 5% (w/v)) and PEO (MW 100,000, 0.25% and 1% (w/v)) concentrations. Target release rate is based on the current dosing regimen of 10 μg/day, and assuming a 1 mL injection of silk hydrogel formulation. Legend: S: Silk, E: Exenatide.

FIG. 5 is a line graph showing Exenatide concentrations in vitro for silk hydrogel formulations with and without BSA. Formulations have different silk (4% and 8%) and exenatide (0.06% and 0.12%) concentrations (w/v), with variations in BSA loading (0 and 5% (w/v)). Target release rate is based on the current dosing regimen of 10 μg/day, and assuming a 1 mL injection of silk hydrogel formulation. Legend: S: Silk, E: Exenatide.

### DETAILED DESCRIPTION OF EXEMPLARY EMBODIMENTS

The present disclosure provides a solution to the problems associated with daily or weekly administration of therapeutic agents for chronic diseases and disorders. The silk-based drug delivery compositions described herein were developed to address the issues associated with repeated injections. In solving this problem, the inventors have demonstrated that the use of silk-based drug delivery compositions for sustained release of exemplary therapeutic agents, GLP-1 receptor agonists (e.g. exenatide and liraglutide), for more than two months in vitro and one week in vivo.

Generally, the silk-based drug delivery composition described herein comprises a therapeutic agent dispersed or encapsulated in a silk matrix. Without limitations, the therapeutic agent can be dispersed homogenously or heterogeneously within the silk matrix. The terms “dispersed” and “encapsulated” are used interchangeably herein when used in reference to presence of the therapeutic agent in the silk matrix.

Without limitations, the silk matrix can have any size, shape, or dimension as desired. For example, the silk matrix can be in the form of a particle, a fiber, a film, a gel, a mesh, a mat, a non-woven mat, a powder, a liquid, or any combinations thereof. In some embodiments, the silk matrix can have a cross-section. Cross-section can be, for example without limitations, round, substantially round, oval, substantially oval, elliptical, substantially elliptical, triangular, substantially triangular, square, substantially square, hexagonal, substantially hexagonal, or the like.

In some embodiments, the silk matrix can be in the form of a hydrogel. As used herein, the term “hydrogel” refers to a swellable polymeric matrix, consisting of a three-dimensional network of macromolecules held together by covalent or non-covalent crosslinks, which can absorb a substantial amount of liquid, e.g., water, within its structure without dissolution. In some embodiments, the silk matrix is in the form of a particle, e.g., a micro- or nano-particle.

As used herein, the phrases “silk matrix” generally refers to a matrix comprising silk. In some embodiments, silk can exclude sericin. In some embodiments, silk can comprise silk fibroin, silk sericin or a combination thereof. The term “silk matrix” refer to a matrix or composition in which silk (or silk fibroin) constitutes at least about 1% (w/v or w/w)(e.g., 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, or more) of the total silk matrix composition. In some embodiments, the silk matrix constitutes at least about 30%, at least about 40%, at least about 50%, at least about 60%, at least about 70%, at least about 80%, at least about 90%, at least about 95%, up to and including 100% or any percentages between about 30% and about 100%, of the total silk matrix composition.

As used herein, the term “silk fibroin” or “fibroin” includes silkworm silk and insect or spider silk protein. See e.g., Lucas et al., Adv. Protein Chem. 1958, 13, 107-242. Any type of silk fibroin can be used according to aspects of the present invention. There are many different types of silk produced by a wide variety of species, including, without limitation: Antheraea mylitta; Antheraea pernyi; Antheraea yamamai; Galleria mellonella; Bombyx mori; Bombyx mandarina; Galleria mellonella; Nephila clavipes; Nephila senegalensis; Gasteracantha mammosa; Argiope aurantia; Araneus diadematus; Latrodectus geometricus; Araneus bicentenarius; Tetragnatha versicolor; Araneus ventricosus; Dolomedes tenebrosus; Euagrus chisoseus; Plectreurys tristis; Argiope trifasciata; and Nephila madagascariensis. Other silks include transgenic silks, genetically engineered silks (recombinant silk), such as silks from bacteria, yeast, mammalian cells, transgenic animals, or transgenic plants, and variants thereof. See for example, WO 97/08315 and U.S. Pat. No. 5,245,012, content of both of which is incorporated herein by reference in its entirety. In some embodiments, silk fibroin can be derived from other sources such as spiders, other silkworms, bees, synthesized silk-like peptides, and bioengineered variants thereof. In some embodiments, silk fibroin can be extracted from a gland of silkworm or transgenic silkworms. See for example, WO2007/098951, content of which is incorporated herein by reference in its entirety.

In some embodiments, the composition comprises low molecular weight silk fibroin fragments, i.e., the composition comprises a population of silk fibroin fragments having a range of molecular weights, characterized in that: no more than 15% of total weight of the silk fibroin fragments in the population has a molecular weight exceeding 200 kDa, and at least 50% of the total weight of the silk fibroin fragments in the population has a molecular weight within a specified range, wherein the specified range is between about 3.5 kDa and about 120 kDa. Without limitations, the molecular weight can be the peak average molecular weight (Mp), the number average molecular weight (Mn), or the weight average molecular weight (Mw)

As used herein, the phrase “silk fibroin fragments” refers to polypeptides having an amino acid sequence corresponding to fragments derived from silk fibroin protein, or variants thereof. In the context of the present disclosure, silk fibroin fragments generally refer to silk fibroin polypeptides that are smaller than the naturally occurring full length silk fibroin counterpart, such that one or more of the silk fibroin fragments within a population or composition are less than 300 kDa, less than 250 kDa, less than 200 kDa, less than 175 kDa, less than 150 kDa, less than 120 kDa, less than 100 kDa, less than 90 kDa, less than 80 kDa, less than 70 kDa, less than 60 kDa, less than 50 kDa, less than 40 kDa, less than 30 kDa, less than 25 kDa, less than 20 kDa, less than 15 kDa, less than 12 kDa, less than 10 kDa, less than 9 kDa, less than 8 kDa, less than 7 kDa, less than 6 kDa, less than 5 kDa, less than 4 kDa, less than 3.5 kDa, etc. In some embodiments, “a composition comprising silk fibroin fragments” encompasses a composition comprising non-fragmented (i.e., full-length) silk fibroin polypeptide, in additional to shorter fragments of silk fibroin polypeptides. Silk fibroin fragments described herein can be produced as recombinant proteins, or derived or isolated (e.g., purified) from a native silk fibroin protein or silk cocoons. In some embodiments, the silk fibroin fragments can be derived by degumming silk cocoons under a specified condition selected to produce the silk fibroin fragments having the desired range of molecular weights. Low molecular weight silk fibroin compositions are described in U.S. Provisional Application Ser. No. 61/883,732, filed on Sep. 27, 2013, content of which is incorporated herein by reference in its entirety.

In some embodiments, the silk fibroin is substantially depleted of its native sericin content (e.g., 5% (w/w) or less residual sericin in the final extracted silk). Alternatively, higher concentrations of residual sericin can be left on the silk following extraction or the extraction step canbe omitted. In some embodiments, the sericin-depleted silk fibroin has, e.g., about 1% (w/w) residual sericin, about 2% (w/w) residual sericin, about 3% (w/w) residual sericin, about 4% (w/w), or about 5% (w/w) residual sericin. In some embodiments, the sericin-depleted silk fibroin has, e.g., at most 1% (w/w) residual sericin, at most 2% (w/w) residual sericin, at most 3% (w/w) residual sericin, at most 4% (w/w), or at most 5% (w/w) residual sericin. In some other embodiments, the sericin-depleted silk fibroin has, e.g., about 1% (w/w) to about 2% (w/w) residual sericin, about 1% (w/w) to about 3% (w/w) residual sericin, about 1% (w/w) to about 4% (w/w), or about 1% (w/w) to about 5% (w/w) residual sericin. In some embodiments, the silk fibroin is entirely free of its native sericin content. As used herein, the term “entirely free” (i.e. “consisting of” terminology) means that within the detection range of the instrument or process being used, the substance cannot be detected or its presence cannot be confirmed. In some embodiments, the silk fibroin is essentially free of its native sericin content. As used herein, the term “essentially free” (or “consisting essentially of) means that only trace amounts of the substance can be detected.

Without wishing to be bound by a theory, properties of the silk-based drug delivery compositions disclosed herein can be modify through controlled partial removal of silk sericin or deliberate enrichment of source silk with sericin. This can be accomplished by varying the conditions, such as time, temperature, concentration, and the like for the silk degumming process.

Degummed silk can be prepared by any conventional method known to one skilled in the art. For example, B. mori cocoons are boiled for about up to 90 minutes, generally about 10 to 60 minutes, in an aqueous solution. In one embodiment, the aqueous solution is about 0.02M Na₂CO₃. The cocoons are rinsed, for example, with water to extract the sericin proteins. The degummed silk can be dried and used for preparing silk powder. Alternatively, the extracted silk can dissolved in an aqueous salt solution. Salts useful for this purpose include lithium bromide, lithium thiocyanate, calcium nitrate or other chemicals capable of solubilizing silk. In some embodiments, the extracted silk can be dissolved in about 8M-12 M LiBr solution. The salt is consequently removed using, for example, dialysis.

If necessary, the solution can then be concentrated using, for example, dialysis against a hygroscopic polymer, for example, PEG, a polyethylene oxide, amylose or sericin. In some embodiments, the PEG is of a molecular weight of 8,000-10,000 g/mol and has a concentration of about 10% to about 50% (w/v). A slide-a-lyzer dialysis cassette (Pierce, MW CO 3500) can be used. However, any dialysis system can be used. The dialysis can be performed for a time period sufficient to result in a final concentration of aqueous silk solution between about 10% to about 30%. In most cases dialysis for 2-12 hours can be sufficient. See, for example, International Patent Application Publication No. WO 2005/012606, the content of which is incorporated herein by reference in its entirety. Another method to generate a concentrated silk solution comprises drying a dilute silk solution (e.g., through evaporation or lyophilization). The dilute solution can be dried partially to reduce the volume thereby increasing the silk concentration. The dilute solution can be dried completely and then dissolving the dried silk fibroin in a smaller volume of solvent compared to that of the dilute silk solution.

In some embodiments, the silk fibroin solution can be produced using organic solvents. Such methods have been described, for example, in Li, M., et al., J. Appl. Poly Sci. 2001, 79, 2192-2199; Min, S., et al. Sen'I Gakkaishi 1997, 54, 85-92; Nazarov, R. et al., Biomacromolecules 2004 5,718-26, content of all which is incorporated herein by reference in their entirety. An exemplary organic solvent that can be used to produce a silk solution includes, but is not limited to, hexafluoroisopropanol (HFIP). See, for example, International Application No. WO2004/000915, content of which is incorporated herein by reference in its entirety. In some embodiments, the silk solution is entirely free or essentially free of organic solvents, i.e., solvents other than water.

Generally, any amount of silk fibroin can be present in the solution used for forming the silk based drug delivery composition. For example, amount of silk fibroin in the solution can be from about 0.1% (w/v) to about 90% (w/v). In some embodiments, the amount of silk fibroin in the solution can be from about 1% (w/v) to about 75% (w/v), from about 1% (w/v) to about 70% (w/v), from about 1% (w/v) to about 65% (w/v), from about 1% (w/v) to about 60% (w/v), from about 1% (w/v) to about 55% (w/v), from about 1% (w/v) to about 50% (w/v), from about 1% (w/v) to about 35% (w/v), from about 1% (w/v) to about 30% (w/v), from about 1% (w/v) to about 25% (w/v), from about 1% (w/v) to about 20% (w/v), from about 1% (w/v) to about 15% (w/v), from about 1% (w/v) to about 10% (w/v), from about 5% (w/v) to about 25% (w/v), from about 5% (w/v) to about 20% (w/v), from about 5% (w/v) to about 15% (w/v). In some embodiments, the silk fibroin in the solution is about 25% (w/v). In some embodiments, the silk fibroin in the solution is about 0.5 (w/v) to about 30% (w/v), about 4% (w/v) to about 16% (w/v), about 4% (w/v) to about 14% (w/v), about 4% (w/v) to about 12% (w/v), about 4% (w/v) to about 0% (w/v), about 6% (w/v) to about 8% (w/v). In some embodiments, the silk fibroin solution has a silk fibroin concentration of from about 5% to about 40%, from 10% to about 40%, or from about 15% to about 40% (w/v). In some embodiments, the silk fibroin solution has a silk fibroin concentration of about 5% (w/v), about 7.5% (w/v), about 8% (w/v), about 10% (w/v), about 12.5% (w/v), about 15% (w/v), about 17.5% (w/v), about 20% (w/v), about 22.5% (w/v), about 25% (w/v), about 27.5% (w/v), about 30% (w/v), about 32.5% (w/v), about 35% (w/v), about 37.5% (w/v), about 40% (w/v), about 42.5% (w/v), about 45% (w/v), about 47.5% (w/v), or about 50% (w/v). Exact amount of silk in the silk solution can be determined by drying a known amount of the silk solution and measuring the mass of the residue to calculate the solution concentration.

Generally, any amount of silk fibroin can be present in the silk-based drug delivery composition disclosed herein. For example, amount of silk fibroin in the silk-based drug delivery composition can be from about 1% (w/w) to about 90% (w/w). In some embodiments, the amount of silk fibroin in the composition can be from about 0.1% (w/w) to about 75% (w/w), from about 1% (w/w) to about 70% (w/w), from about 1% (w/w) to about 65% (w/w), from about 1% (w/w) to about 60% (w/w), from about 1% (w/w) to about 55% (w/w), from about 1% (w/w) to about 50% (w/w), from about 1% (w/w) to about 45% (w/w), from about 1% (w/w) to about 40% (w/w), from about 1% (w/w) to about 35% (w/w), from about 1% (w/w) to about 30% (w/w), from about 1% (w/w) to about 25% (w/w), from about 1% (w/w) to about 20% (w/w), from about 1% (w/w) to about 15% (w/w), from about 1% (w/w) to about 10% (w/w), from about 5% (w/w) to about 25% (w/w), from about 5% (w/w) to about 20% (w/w), from about 5% (w/w) to about 15% (w/w). In some embodiments, the silk fibroin in the composition is about 25% (w/w). In some embodiments, the silk in the composition is about 0.5 (w/w) to about 30% (w/w), about 2% (w/w) to about 8% (w/w), about 2% (w/w) to about 7% (w/w), about 2% (w/w) to about 6% (w/w), about 2% (w/w) to about 5% (w/w), about 3% (w/w) to about 4% (w/w).

Without wishing to be bound by a theory, molecular weight of silk or the silk fibroin concentration used for preparing the silk matrix can have an effect on properties of the silk matrix, such as swelling ratio, degradation, drug release kinetics and the like

Depending on the desired mechanical property of a silk matrix, and/or release profile of the therapeutic agent from the silk matrix, different material states or forms of the silk matrix can be produced. For example, the silk matrix can be produced in a form of a hydrogel, a microparticle, a nanoparticle, a fiber, a film, lyophilized powder, a lyophilized gel, a reservoir implant, a homogenous implant, a gel-like or gel particle, and any combinations thereof. Accordingly, different concentrations of silk fibroin can be included in the silk matrix to achieve different material states or forms.

In some embodiments, the silk matrix encapsulating a therapeutic agent can be in a form of a hydrogel. Exemplary methods for preparing silk fibroin gels and hydrogels include, but are not limited to, sonication, vortexing, pH titration, exposure to electric field, solvent immersion, water annealing, water vapor annealing, and the like. Exemplary methods for preparing silk fibroin gels and hydrogels are described in, for example, WO 2005/012606, WO 2008/150861, WO 2010/036992, and WO 2011/005381; and U.S. Pat. App. Pub No. U.S. 2010/0178304 and No.: US 2011/0171239, content of all of which is incorporated herein by reference in its entirety. Gels formed by exposure to electric field are also referred to as e-gels herein. Methods for forming e-gels are described in, for example, US2011/0171239, content of which is incorporated herein by reference in its entirety.

In some embodiments, the silk matrix can be in the form of a sponge or foam. In some embodiments, the foam or sponge is a patterned foam or sponge, e.g., nanopatterned foam or sponge. Exemplary methods for preparing silk foams and sponges are described in, for example, WO 2004/000915, WO 2004/000255, and WO 2005/012606, content of all of which is incorporated herein by reference in its entirety.

In some embodiments, the silk matrix can be in the form of a cylindrical matrix, e.g., a silk tube. The silk tubes can be made using any method known in the art. For example, tubes can be made using molding, dipping, electrospinning, gel spinning, and the like. Gel spinning is described in Lovett et al. (Biomaterials 2008, 29(35):4650-4657) and the construction of gel-spun silk tubes is described in PCT application no. PCT/US2009/039870, filed Apr. 8, 2009, contents of both of which are incorporated herein by reference in their entireties Construction of silk tubes using the dip-coating method is described in PCT application no. PCT/US2008/072742, filed Aug. 11, 2008, content of which is incorporated herein by reference in its entirety. Construction of silk fibroin tubes using film-spinning is described in PCT application No. PCT/US2013/030206, filed Mar. 11, 2013 and U.S. Provisional application No. 61/613,185, filed Mar. 20, 2012, contents of both of which are incorporated herein by reference in their entireties.

In some embodiments, the silk matrix can be in the form of a film, e.g., a silk film. As used herein, the term “film” refers to a flat or tubular flexible structure. It is to be noted that the term “film” is used in a generic sense to include a web, film, sheet, laminate, or the like. In some embodiments, the film is a patterned film, e.g., nanopatterned film. Exemplary methods for preparing silk fibroin films are described in, for example, WO 2004/000915 and WO 2005/012606, content of both of which is incorporated herein by reference in its entirety.

In some embodiments, the silk matrix can be in the form of a fiber. As used herein, the term “fiber” means a relatively flexible, unit of matter having a high ratio of length to width across its cross-sectional perpendicular to its length. Methods for preparing silk fibroin fibers are well known in the art. A fiber can be prepared by electrospinning a silk solution, drawing a silk solution, and the like. Electrospun silk materials, such as fibers, and methods for preparing the same are described, for example in WO2011/008842, content of which is incorporated herein by reference in its entirety. Micron-sized silk fibers (e.g., 10-600 μm in size) and methods for preparing the same are described, for example in Mandal et al., PNAS, 2012, doi: 10.1073/pnas.1119474109; U.S. Provisional Application No. 61/621,209, filed Apr. 6, 2012; and PCT application no. PCT/US13/35389, filed Apr. 5, 2013, contents of all of which are incorporated herein by reference in their entireties.

In some embodiments where the silk hydrogel having a high silk concentration, e.g., a concentration too high for injection such as a silk or silk fibroin concentration of at least about 5% (w/v), at least about 8% (w/v), at least about 10% (w/v), at least about 15% (w/v), at least about 20% (w/v), at least about 30% (w/v) or higher, the silk hydrogel can be reduced into gel-like or gel particles, e.g., by grinding, cutting, crushing, sieving, sifting, and/or filtering. Without limitations, the gel-like or gel particles can be of any size suitable for injection, e.g., a size of about 0.5 μm to about 2 mm, about 1 μm to about 1 mm, about 10 μm to about 0.5 mm, or about 50 μm to about 0.1 mm. In some embodiments, the gel-like or gel particles can have a size ranging from about 0.01 μm to about 1000 μm, about 0.05 μm to about 500 μm, about 0.1 μm to about 250 μm, about 0.25 μm to about 200 μm, or about 0.5 μm to about 100 μm.

Accordingly, in some embodiments, the silk matrix encapsulating a therapeutic agent can be in the form of a particle. When the silk matrix encapsulating the therapeutic agent is in the form of a particle, the particle can be of any shape or form, e.g., spherical, rod, elliptical, cylindrical, capsule, or disc.

In some embodiments, the particle is a microparticle or a nanoparticle. As used herein, the term “microparticle” refers to a particle having a particle size of about 0.01 μm to about 1000 μm. In some embodiments, the microparticle as a size of about 0.05 μm to about 750 nm, about 0.1 μm to about 500 μm, about 0.25 μm to about 250 μm, or about 0.5 μm to about 100 μm. In one embodiment, the microparticle has a particle size of about 75 μm. As used herein, the term “nanoparticle” refers to particle having a particle size of about 0.1 μm to about 1000 μm. For example, a nanoparticle can have a particle size of about 0.5 μm to about 500 μm, about 1 μm to about 250 μm, about 10 μm to about 150 μm, or about 15 μm to about 100 μm.

It will be understood by one of ordinary skill in the art that microparticles or nanoparticles usually exhibit a distribution of particle sizes around the indicated “size.” Unless otherwise stated, the term “size” as used herein refers to the mode of a size distribution of microparticles or nanoparticles, i.e., the value that occurs most frequently in the size distribution. Methods for measuring the microparticle or nanoparticle size are known to a skilled artisan, e.g., by dynamic light scattering (such as photocorrelation spectroscopy, laser diffraction, low-angle laser light scattering (LALLS), and medium-angle laser light scattering (MALLS)), light obscuration methods (such as Coulter analysis method), or other techniques (such as rheology, and light or electron microscopy).

When the silk matrix comprising the therapeutic agent is in the form of a particle, the particle can be substantially spherical. What is meant by “substantially spherical” is that the ratio of the lengths of the longest to the shortest perpendicular axes of the particle cross section is less than or equal to about 1.5. Substantially spherical does not require a line of symmetry. Further, the particles can have surface texturing, such as lines or indentations or protuberances that are small in scale when compared to the overall size of the particle and still be substantially spherical. In some embodiments, the ratio of lengths between the longest and shortest axes of the particle is less than or equal to about 1.5, less than or equal to about 1.45, less than or equal to about 1.4, less than or equal to about 1.35, less than or equal to about 1.30, less than or equal to about 1.25, less than or equal to about 1.20, less than or equal to about 1.15 less than or equal to about 1.1. Without wishing to be bound by a theory, surface contact is minimized in particles that are substantially spherical, which minimizes the undesirable agglomeration of the particles upon storage. Many crystals or flakes have flat surfaces that can allow large surface contact areas where agglomeration can occur by ionic or non-ionic interactions. A sphere permits contact over a much smaller area.

In some embodiments, the particles have substantially the same particle size. Particles having a broad size distribution where there are both relatively big and small particles allow for the smaller particles to fill in the gaps between the larger particles, thereby creating new contact surfaces. A broad size distribution can result in larger spheres by creating many contact opportunities for binding agglomeration. The particles described herein are within a narrow size distribution, thereby minimizing opportunities for contact agglomeration. What is meant by a “narrow size distribution” is a particle size distribution that has a ratio of the volume diameter of the 90th percentile of the small spherical particles to the volume diameter of the 10th percentile less than or equal to 5. In some embodiments, the volume diameter of the 90th percentile of the small spherical particles to the volume diameter of the 10th percentile is less than or equal to 4.5, less than or equal to 4, less than or equal to 3.5, less than or equal to 3, less than or equal to 2.5, less than or equal to 2, less than or equal to 1.5, less than or equal to 1.45, less than or equal to 1.40, less than or equal to 1.35, less than or equal to 1.3, less than or equal to 1.25, less than or equal to 1.20, less than or equal to 1.15, or less than or equal to 1.1.

Geometric Standard Deviation (GSD) can also be used to indicate the narrow size distribution. GSD calculations involved determining the effective cutoff diameter (ECD) at the cumulative less than percentages of 15.9% and 84.1%. GSD is equal to the square root of the ratio of the ECD less than 84.17% to ECD less than 15.9%. The GSD has a narrow size distribution when GSD<2.5. In some embodiments, GSD is less than 2, less than 1.75, or less than 1.5. In one embodiment, GSD is less than 1.8.

Various methods of producing silk microparticles or nanoparticles are known in the art. In some embodiments, the silk microparticles or nanoparticles can be produced by a polyvinyl alcohol (PVA) phase separation method as described in, e.g., International App. No. WO 2011/041395, the content of which is incorporated herein by reference in its entirety. Other methods for producing silk microparticles or nanoparticles are described in, for example, U.S. App. No. U.S. 2010/0028451 and International App. No.: WO 2008/118133 (using lipid as a template for making silk microspheres or nanospheres); and in Wenk et al. J Control Release 2008; 132: 26-34 (using spraying method to produce silk microspheres or nanospheres), contents of all which are incorporated herein by reference in their entireties. Certain embodiments of micro- to nano-scale silk fibroin particles and related technology are also provided in U.S. Provisional Application Ser. No. 61/883,933, filed Sep. 27, 2013, titled “SYNTHESIS OF SILK FIBROIN MICRO- AND SUBMICRON SPHERES USING A CO-FLOW METHOD,” content of which is incorporated herein by reference in its entirety.

In some embodiments, silk particles can be produced using a freeze-drying method as described in U.S. Provisional Application Ser. No. 61/719,146, filed Oct. 26, 2012, content of which is incorporated herein by reference in its entirety. Specifically, silk foam can be produced by freeze-drying a silk solution. The foam then can be reduced to particles. For example, a silk solution can be cooled to a temperature at which the liquid carrier transforms into a plurality of solid crystals or particles and removing at least some of the plurality of solid crystals or particles to leave a porous silk material (e.g., silk foam). After cooling, liquid carrier can be removed, at least partially, by sublimation, evaporation, and/or lyophilization. In some embodiments, the liquid carrier can be removed under reduced pressure. After formation, the silk fibroin foam can be subjected to grinding, cutting, crushing, or any combinations thereof to form silk particles. For example, the silk fibroin foam can be blended in a conventional blender or milled in a ball mill to form silk particles of desired size.

In some embodiments, the silk matrix comprising the therapeutic agent can be lyophilized or freeze-dried.

Optionally, the conformation of the silk fibroin in the silk matrix can be altered after formation of the silk matrix. Without wishing to be bound by a theory, the induced conformational change can alter the crystallinity of the silk fibroin in the silk matrix, e.g., Silk II beta-sheet crystallinity. This can alter the rate of release of the therapeutic agent from the silk matrix. The conformational change can be induced by any methods known in the art, including, but not limited to, alcohol immersion (e.g., ethanol, methanol), water annealing, shear stress, ultrasound (e.g., by sonication), pH reduction (e.g., pH titration and/or exposure to an electric field) and any combinations thereof. For example, the conformational change can be induced by one or more methods, including but not limited to, controlled slow drying (Lu et al., Biomacromolecules 2009, 10, 1032); water annealing (Jin et al., 15 Adv. Funct. Mats. 2005, 15, 1241; Hu et al., Biomacromolecules 2011, 12, 1686); stretching (Demura & Asakura, Biotech & Bioengin. 1989, 33, 598); compressing; solvent immersion, including methanol (Hofmann et al., J Control Release. 2006, 111, 219), ethanol (Miyairi et al., J. Fermen. Tech. 1978, 56, 303), glutaraldehyde (Acharya et al., Biotechnol J. 2008, 3, 226), and 1-ethyl-3-β-dimethyl aminopropyl) carbodiimide (EDC) (Bayraktar et al., Eur J Pharm Biopharm. 2005, 60, 373); pH adjustment, e.g., pH titration and/or exposure to an electric field (see, e.g., U.S. Patent App. No. US2011/0171239); heat treatment; shear stress (see, e.g., International App. No.: WO 2011/005381), ultrasound, e.g., sonication (see, e.g., U.S. Patent Application Publication No. U.S. 2010/0178304 and International App. No. WO2008/150861); and any combinations thereof. Content of all of the references listed above is incorporated herein by reference in their entirety.

In some embodiments, the conformation of the silk fibroin can be altered by water annealing. Without wishing to be bound by a theory, it is believed that physical temperature-controlled water vapor annealing (TCWVA) provides a simple and effective method to obtain refined control of the molecular structure of silk biomaterials. The silk materials can be prepared with control of crystallinity, from a low content using conditions at 4° C. (α helix dominated silk I structure), to highest content of ˜60% crystallinity at 100° C. (β-sheet dominated silk II structure). This physical approach covers the range of structures previously reported to govern crystallization during the fabrication of silk materials, yet offers a simpler, green chemistry, approach with tight control of reproducibility. Temperature controlled water vapor annealing is described, for example, in Hu et al., Biomacromolecules, 2011, 12, 1686-1696, content of which is incorporated herein by reference in its entirety.

In some embodiments, alteration in the conformation of the silk fibroin can be induced by immersing in alcohol, e.g., methanol, ethanol, etc. The alcohol concentration can be at least 10%, at least 20%, at least 30%, at least 40%, at least 50%, at least 60%, at least 70%, at least 80%, at least 90% or 100%. In some embodiment, alcohol concentration is 100%. If the alteration in the conformation is by immersing in a solvent, the silk composition can be washed, e.g., with solvent/water gradient to remove any of the residual solvent that is used for the immersion. The washing can be repeated one, e.g., one, two, three, four, five, or more times.

Alternatively, the alteration in the conformation of the silk fibroin can be induced with sheer stress. The sheer stress can be applied, for example, by passing the silk composition through a needle. Other methods of inducing conformational changes include applying an electric field, applying pressure, or changing the salt concentration.

The treatment time for inducing the conformational change can be any period of time to provide a desired silk II (beta-sheet crystallinity) content. In some embodiments, the treatment time can range from about 1 hour to about 12 hours, from about 1 hour to about 6 hours, from about 1 hour to about 5 hours, from about 1 hour to about 4 hours, or from about 1 hour to about 3 hours. In some embodiments, the sintering time can range from about 2 hours to about 4 hours or from 2.5 hours to about 3.5 hours.

When inducing the conformational change is by solvent immersion, treatment time can range from minutes to hours. For example, immersion in the solvent can be for a period of at least about 15 minutes, at least about 30 minutes, at least about 1 hour, at least about 2 hours, at least 3 hours, at least about 6 hours, at least about 18 hours, at least about 12 hours, at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, or at least about 14 days. In some embodiments, immersion in the solvent can be for a period of about 12 hours to about seven days, about 1 day to about 6 days, about 2 to about 5 days, or about 3 to about 4 days.

After the treatment to induce the conformational change, silk fibroin can comprise a silk II beta-sheet crystallinity content of at least about 5%, at least about 10%, at least about 20%, at least about 30%, at least about 40%, at least about 50%, at least about 60%, at least about 70%, at least about 80%, at least about 90%, or at least about 95% but not 100% (i.e., all the silk is present in a silk II beta-sheet conformation). In some embodiments, silk is present completely in a silk II beta-sheet conformation, i.e., 100% silk II beta-sheet crystallinity.

In some embodiments, the silk fibroin in the composition has a protein structure that substantially includes β-turn and β-strand regions. Without wishing to be bound by a theory, the silk 3 sheet content can impact function and in vivo longevity of the composition. It is to be understood that composition including non-β sheet content (e.g., e-gels) can also be utilized. In aspects of these embodiments, the silk fibroin in the composition has a protein structure including, e.g., about 10% β-turn and β-strand regions, about 20% β-turn and β-strand regions, about 30% β-turn and β-strand regions, about 40% β-turn and β-strand regions, about 50% β-turn and β-strand regions, about 60% β-turn and β-strand regions, about 70% β-turn and β-strand regions, about 80% β-turn and β-strand regions, about 90% β-turn and β-strand regions, or about 100% β-turn and β-strand regions. In other aspects of these embodiments, the silk fibroin in the composition has a protein structure including, e.g., at least 10% β-turn and β-strand regions, at least 20% β-turn and β-strand regions, at least 30% β-turn and β-strand regions, at least 40% β-turn and β-strand regions, at least 50% β-turn and β-strand regions, at least 60% β-turn and β-strand regions, at least 70% β-turn and β-strand regions, at least 80% β-turn and β-strand regions, at least 90% β-turn and β-strand regions, or at least 95% β-turn and β-strand regions. In yet other aspects of these embodiments, the silk fibroin in the composition has a protein structure including, e.g., about 10% to about 30% β-turn and β-strand regions, about 20% to about 40% β-turn and β-strand regions, about 30% to about 50% β-turn and β-strand regions, about 40% to about 60% β-turn and β-strand regions, about 50% to about 70% β-turn and β-strand regions, about 60% to about 80% β-turn and β-strand regions, about 70% to about 90% β-turn and β-strand regions, about 80% to about 100% β-turn and β-strand regions, about 10% to about 40% β-turn and β-strand regions, about 30% to about 60% β-turn and β-strand regions, about 50% to about 80% β-turn and β-strand regions, about 70% to about 100% β-turn and β-strand regions, about 40% to about 80% β-turn and β-strand regions, about 50% to about 90% β-turn and β-strand regions, about 60% to about 100% β-turn and β-strand regions, or about 50% to about 100% β-turn and β-strand regions. In some embodiments, silk 3 sheet content, from less than 10% to ˜55% can be used in the silk-based drug delivery composition.

In some embodiments, the silk fibroin in the composition has a protein structure that is substantially-free of α-helix and random coil regions. In aspects of these embodiments, the silk fibroin in the composition has a protein structure including, e.g., about 5% α-helix and random coil regions, about 10% α-helix and random coil regions, about 15% α-helix and random coil regions, about 20% α-helix and random coil regions, about 25% α-helix and random coil regions, about 30% α-helix and random coil regions, about 35% α-helix and random coil regions, about 40% α-helix and random coil regions, about 45% α-helix and random coil regions, or about 50% α-helix and random coil regions. In other aspects of these embodiments, the silk fibroin in the composition has a protein structure including, e.g., at most 5% α-helix and random coil regions, at most 10% α-helix and random coil regions, at most 15% α-helix and random coil regions, at most 20% α-helix and random coil regions, at most 25% α-helix and random coil regions, at most 30% α-helix and random coil regions, at most 35% α-helix and random coil regions, at most 40% α-helix and random coil regions, at most 45% α-helix and random coil regions, or at most 50% α-helix and random coil regions. In yet other aspects of these embodiments, the silk fibroin in the composition has a protein structure including, e.g., about 5% to about 10% α-helix and random coil regions, about 5% to about 15% α-helix and random coil regions, about 5% to about 20% α-helix and random coil regions, about 5% to about 25% α-helix and random coil regions, about 5% to about 30% α-helix and random coil regions, about 5% to about 40% α-helix and random coil regions, about 5% to about 50% α-helix and random coil regions, about 10% to about 20% α-helix and random coil regions, about 10% to about 30% α-helix and random coil regions, about 15% to about 25% α-helix and random coil regions, about 15% to about 30% α-helix and random coil regions, or about 15% to about 35% α-helix and random coil regions.

In some embodiments, the silk fibroin in the composition has a protein structure that substantially includes β-turn and β-strand regions. In aspects of these embodiments, the silk fibroin in the composition has a protein structure including, e.g., about 10% β-turn and β-strand regions, about 20% β-turn and β-strand regions, about 30% β-turn and β-strand regions, about 40% β-turn and β-strand regions, about 50% β-turn and β-strand regions, about 60% β-turn and β-strand regions, about 70% β-turn and β-strand regions, about 80% β-turn and β-strand regions, about 90% β-turn and β-strand regions, or about 100% β-turn and β-strand regions. In other aspects of these embodiments, the silk fibroin in the composition has a protein structure including, e.g., at least 10% β-turn and β-strand regions, at least 20% β-turn and β-strand regions, at least 30% β-turn and β-strand regions, at least 40% β-turn and β-strand regions, at least 50% β-turn and β-strand regions, at least 60% β-turn and β-strand regions, at least 70% β-turn and β-strand regions, at least 80% β-turn and β-strand regions, at least 90% β-turn and β-strand regions, or at least 95% β-turn and β-strand regions. In yet other aspects of these embodiments, the silk fibroin in the composition has a protein structure including, e.g., about 10% to about 30% β-turn and β-strand regions, about 20% to about 40% β-turn and β-strand regions, about 30% to about 50% β-turn and β-strand regions, about 40% to about 60% β-turn and β-strand regions, about 50% to about 70% β-turn and β-strand regions, about 60% to about 80% β-turn and β-strand regions, about 70% to about 90% β-turn and β-strand regions, about 80% to about 100% β-turn and β-strand regions, about 10% to about 40% β-turn and β-strand regions, about 30% to about 60% β-turn and β-strand regions, about 50% to about 80% β-turn and β-strand regions, about 70% to about 100% β-turn and β-strand regions, about 40% to about 80% β-turn and β-strand regions, about 50% to about 90% β-turn and β-strand regions, about 60% to about 100% β-turn and β-strand regions, or about 50% to about 100% β-turn and β-strand regions.

In some embodiments, the silk fibroin in the composition has a protein structure that is substantially-free of α-helix and random coil regions. In aspects of these embodiments, the silk fibroin in the composition has a protein structure including, e.g., about 5% α-helix and random coil regions, about 10% α-helix and random coil regions, about 15% α-helix and random coil regions, about 20% α-helix and random coil regions, about 25% α-helix and random coil regions, about 30% α-helix and random coil regions, about 35% α-helix and random coil regions, about 40% α-helix and random coil regions, about 45% α-helix and random coil regions, or about 50% α-helix and random coil regions. In other aspects of these embodiments, the silk fibroin in the composition has a protein structure including, e.g., at most 5% α-helix and random coil regions, at most 10% α-helix and random coil regions, at most 15% α-helix and random coil regions, at most 20% α-helix and random coil regions, at most 25% α-helix and random coil regions, at most 30% α-helix and random coil regions, at most 35% α-helix and random coil regions, at most 40% α-helix and random coil regions, at most 45% α-helix and random coil regions, or at most 50% α-helix and random coil regions. In yet other aspects of these embodiments, the silk fibroin in the composition has a protein structure including, e.g., about 5% to about 10% α-helix and random coil regions, about 5% to about 15% α-helix and random coil regions, about 5% to about 20% α-helix and random coil regions, about 5% to about 25% α-helix and random coil regions, about 5% to about 30% α-helix and random coil regions, about 5% to about 40% α-helix and random coil regions, about 5% to about 50% α-helix and random coil regions, about 10% to about 20% α-helix and random coil regions, about 10% to about 30% α-helix and random coil regions, about 15% to about 25% α-helix and random coil regions, about 15% to about 30% α-helix and random coil regions, or about 15% to about 35% α-helix and random coil regions.

In some embodiments, the silk fibroin can be modified for different applications and/or desired mechanical or chemical properties (e.g., to facilitate formation of a gradient of a therapeutic agent in silk fibroin matrices). One of skill in the art can select appropriate methods to modify silk fibroins, e.g., depending on the side groups of the silk fibroins, desired reactivity of the silk fibroin and/or desired charge density on the silk fibroin. In one embodiment, modification of silk fibroin can use the amino acid side chain chemistry, such as chemical modifications through covalent bonding, or modifications through charge-charge interaction. Exemplary chemical modification methods include, but are not limited to, carbodiimide coupling reaction (see, e.g., U.S. Patent Application. No. US 2007/0212730), diazonium coupling reaction (see, e.g., U.S. Patent Application No. US 2009/0232963), avidin-biotin interaction (see, e.g., International Application No.: WO 2011/011347) and pegylation with a chemically active or activated derivatives of the PEG polymer (see, e.g., International Application No. WO 2010/057142).

Silk fibroin can also be modified through gene modification to alter functionalities of the silk protein (see, e.g., International Application No. WO 2011/006133). For instance, the silk fibroin can be genetically modified, which can provide for further modification of the silk such as the inclusion of a fusion polypeptide comprising a fibrous protein domain and a mineralization domain, which can be used to form an organic-inorganic composite. See WO 2006/076711. In some embodiments, the silk fibroin can be genetically modified to be fused with a protein, e.g., a therapeutic protein. Additionally, the silk fibroin matrix can be combined with a chemical, such as glycerol, that, e.g., affects flexibility and/or solubility of the matrix. See, e.g., WO 2010/042798, Modified Silk films Containing Glycerol.

In some embodiments, the silk fibroin can be modified with positively/negatively charged peptides or polypeptides, such poly-lysine and poly-glutamic acid. While possible, it is not required that every single silk fibroin molecule in the composition be modified with a positively/negatively charged molecule. Methods of derivatizing or modifying silk fibroin with charged molecules are described in, for example, PCT application no. PCT/US2011/027153, filed Mar. 4, 2011, content of which is incorporated herein by reference in its entirety.

Ratio of modified silk fibroin to unmodified silk fibroin can be adjusted to optimize one or more desired properties of the composition, such as drug release rate or kinetics, degradation rate, and the like. Accordingly, in some embodiments, ratio of modified to unmodified silk fibroin in the composition can range from about 1000:1 (w/w) to about 1:1000 (w/w), from about 500:1 (w/w) to about 1:500 (w/w), from about 250:1 (w/w) to about 1:250 (w/w), from about 200:1 (w/w) to about 1:200 (w/w), from about 25:1 (w/w) to about 1:25 (w/w), from about 20:1 (w/w) to about 1:20 (w/w), from about 10:1 (w/w) to about 1:10 (w/w), or from about 5:1 (w/w) to about 1:5 (w/w).

In some embodiments, the composition comprises a molar ratio of modified to unmodified silk fibroin of, e.g., at least 1000:1, at least 900:1, at least 800:1, at least 700:1, at least 600:1, at least 500:1, at least 400:1, at least 300:1, at least 200:1, at least 100:1, at least 90:1, at least 80:1, at least 70:1, at least 60:1, at least 50:1, at least 40:1, at least 30:1, at least 20:1, at least 10:1, at least 7:1, at least 5:1, at least 3:1, at least 1:1, at least 1:3, at least 1:5, at least 1:7, at least 1:10, at least 1:20, at least 1:30, at least 1:40, at least 1:50, at least 1:60, at least 1:70, at least 1:80, at least 1:90, at least 1:100, at least 1:200, at least 1:300, at least 1:400, at least 1:500, at least 600, at least 1:700, at least 1:800, at least 1:900, or at least 1:100.

In some embodiments, the composition comprises a molar ratio of modified to unmodified silk fibroin of, e.g., at most 1000:1, at most 900:1, at most 800:1, at most 700:1, at most 600:1, at most 500:1, at most 400:1, at most 300:1, at most 200:1, 100:1, at most 90:1, at most 80:1, at most 70:1, at most 60:1, at most 50:1, at most 40:1, at most 30:1, at most 20:1, at most 10:1, at most 7:1, at most 5:1, at most 3:1, at most 1:1, at most 1:3, at most 1:5, at most 1:7, at most 1:10, at most 1:20, at most 1:30, at most 1:40, at most 1:50, at most 1:60, at most 1:70, at most 1:80, at most 1:90, at most 1:100, at most 1:200, at most 1:300, at most 1:400, at most 1:500, at most 1:600, at most 1:700, at most 1:800, at most 1:900, or at most 1:1000.

In some embodiments, the composition comprises a molar ratio of modified to unmodified silk fibroin of e.g., from about 1000:1 to about 1:1000, from about 900:1 to about 1:900, from about 800:1 to about 1:800, from about 700:1 to about 1:700, from about 600:1 to about 1:600, from about 500:1 to about 1:500, from about 400:1 to about 1:400, from about 300:1 to about 1:300, from about 200:1 to about 1:200, from about 100:1 to about 1:100, from about 90:1 to about 1:90, from about 80:1 to about 1:80, from about 70:1 to about 1:70, from about 60:1 to about 1:60, from about 50:1 to about 1:50, from about 40:1 to about 1:40, from about 30:1 to about 1:30, from about 20:1 to about 1:20, from about 10:1 to about 1:10, from about 7:1 to about 1:7, from about 5:1 to about 1:5, from about 3:1 to about 1:3, or about 1:1.

The silk-based drug delivery composition can also comprise a targeting ligand. As used herein, the term “targeting ligand” refers to any material or substance which can promote targeting of the drug delivery composition to tissues and/or receptors in vivo and/or in vitro. The targeting ligand can be synthetic, semi-synthetic, or naturally-occurring. Materials or substances which can serve as targeting ligands include, for example, proteins, including antibodies, antibody fragments, hormones, hormone analogues, glycoproteins and lectins, peptides, polypeptides, amino acids, sugars, saccharides, including monosaccharides and polysaccharides, carbohydrates, vitamins, steroids, steroid analogs, hormones, cofactors, and genetic material, including nucleosides, nucleotides, nucleotide acid constructs, petptide nucleic acids (PNA), aptamers, and polynucleotides. Other targeting ligands in the present disclsoure include cell adhesion molecules (CAM), among which are, for example, cytokines, integrins, cadherins, immunoglobulins and selectin. The silk drug delivery composition can also encompass precursor targeting ligands. A precursor to a targeting ligand refers to any material or substance which can be converted to a targeting ligand. Such conversion can involve, for example, anchoring a precursor to a targeting ligand. Exemplary targeting precursor moieties include maleimide groups, disulfide groups, such as ortho-pyridyl disulfide, vinylsulfone groups, azide groups, and [agr]-iodo acetyl groups.

The targeting ligand can be covalently (e.g., cross-linked) or non-covalently linked to the silk-based drug delivery composition. For example, a targeting ligand can be covalently linked to silk fibroin used for making the silk-based drug delivery composition. Alternatively or in addition, a targeting ligand can be linked to an additive present in the silk fibroin solution which is used for making the silk-based drug delivery composition.

In some embodiments, the silk matrix can be porous, wherein the silk matrix can have a porosity of at least about 30%, at least about 40%, at least about 50%, at least about 60%, at least about 70%, at least about 80%, at least about 90%, or higher. Too high porosity can yield a silk matrix with lower mechanical properties, but with faster release of a therapeutic agent. However, too low porosity can decrease the release of a therapeutic agent. One of skill in the art can adjust the porosity accordingly, based on a number of factors such as, but not limited to, desired release rates, molecular size and/or diffusion coefficient of the therapeutic agent, and/or concentrations and/or amounts of silk fibroin in a silk matrix. The term “porosity” as used herein is a measure of void spaces in a material, e.g., a matrix such as silk fibroin, and is a fraction of volume of voids over the total volume, as a percentage between 0 and 100% (or between 0 and 1). Determination of matrix porosity is well known to a skilled artisan, e.g., using standardized techniques, such as mercury porosimetry and gas adsorption, e.g., nitrogen adsorption.

The porous silk matrix can have any pore size. In some embodiments, the pores of a silk matrix can have a size distribution ranging from about 50 nm to about 1000 μm, from about 250 nm to about 500 μm, from about 500 nm to about 250 μm, from about 1 μm to about 200 μm, from about 10 μm to about 150 μm, or from about 50 μm to about 100 μm. As used herein, the term “pore size” refers to a diameter or an effective diameter of the cross-sections of the pores. The term “pore size” can also refer to an average diameter or an average effective diameter of the cross-sections of the pores, based on the measurements of a plurality of pores. The effective diameter of a cross-section that is not circular equals the diameter of a circular cross-section that has the same cross-sectional area as that of the non-circular cross-section. In some embodiments, the silk fibroin can be swollen when the silk fibroin scaffold is hydrated. The sizes of the pores or the mesh size can then change depending on the water content in the silk fibroin. The pores can be filled with a fluid such as water or air.

Methods for forming pores in a silk matrix are known in the art, e.g., porogen-leaching method, freeze-drying method, and/or gas-forming method. Such methods are described, e.g., in U.S. Pat. App. Nos.: US 2010/0279112, US 2010/0279112, and U.S. Pat. No. 7,842,780, the contents of which are incorporated herein by reference in their entirety.

Methods for forming pores in silk fibroin-based scaffolds are known in the art and include, but are not limited, porogen-leaching methods, freeze-drying methods, and/or gas-forming method. Exemplary methods for forming pores in a silk-based material are described, for example, in U.S. Pat. App. Pub. Nos.: US 2010/0279112 and US 2010/0279112; U.S. Pat. No. 7,842,780; and PCT application publication no. WO2004062697, contents of all of which are incorporated herein by reference in their entireties.

Accordingly, any desirable release rates or profiles of a therapeutic agent from a silk matrix can be, at least partly, adjusted by varying silk processing methods, e.g., concentration of silk in a silk matrix, amount of silk fibroin and/or beta-sheet conformation structures in a silk matrix, porosity and/or pore sizes of the silk matrix, and any combinations thereof.

Without limitations, silk-based drug delivery composition can comprise any amount of silk, e.g., silk fibroin. For example, the silk-based drug delivery composition can comprise from about 1% (w/v) to about 50% (w/v) of silk, e.g., silk fibroin. In some embodiments, the silk-based drug delivery composition can comprise from about 1% (w/v) to about 30% (w/v), from about 1% (w/v) to about 25% (w/v), from about 1% (w/v) to about 20% (w/v), from about 1% (w/v) to about 15% (w/v), from about 1% (w/v) to about 10% (w/v), from about 5% (w/v) to about 25% (w/v), from about 5% (w/v) to about 20% (w/v), from about 5% (w/v) to about 15% (w/v) of silk, e.g., silk fibroin. In some embodiments, the silk-based drug delivery composition can comprise from about 2% (w/v) to about 32% (w/v), from about 4% (w/v) to about 16% (w/v), from about 2% (w/v) to about 32% (w/v), or from about 2% (w/v) to about 16% (w/v) of silk, e.g., silk fibroin. In some embodiments, the silk-based drug delivery composition can comprise about 2% (w/v), about 4% (w/v), about 8% (w/v), about 10% (w/v), or about 16% (w/v) of silk.

Generally, any therapeutic agent can be encapsulated in the silk based drug delivery compositions described herein. As used herein, the term “therapeutic agent” means a molecule, group of molecules, complex or substance administered to an organism for diagnostic, therapeutic, preventative medical, or veterinary purposes. As used herein, the term “therapeutic agent” includes a “drug” or a “vaccine.” This term include externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term can also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a therapeutic effect, for example deoxyribonucleic acid (DNA), ribonucleic acid (RNA), or mixtures or combinations thereof, including, for example, DNAnanoplexes, siRNA, shRNA, aptamers, ribozymes, decoy nucleic acids, antisense nucleic acids, RNA activators, and the like.

The term “therapeutic agent” also includes an agent that is capable of providing a local or systemic biological, physiological, or therapeutic effect in the biological system to which it is applied. For example, the therapeutic agent can act to control infection or inflammation, enhance cell growth and tissue regeneration, control tumor growth, act as an analgesic, promote anti-cell attachment, and enhance bone growth, among other functions. Other suitable therapeutic agents can include anti-viral agents, hormones, antibodies, or therapeutic proteins. Other therapeutic agents include prodrugs, which are agents that are not biologically active when administered but, upon administration to a subject are converted to biologically active agents through metabolism or some other mechanism. Additionally, a silk-based drug delivery composition can contain combinations of two or more therapeutic agents.

A therapeutic agent can include a wide variety of different compounds, including chemical compounds and mixtures of chemical compounds, e.g., small organic or inorganic molecules; saccharines; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; an extract made from biological materials such as bacteria, plants, fungi, or animal cells; animal tissues; naturally occurring or synthetic compositions; and any combinations thereof. In some embodiments, the therapeutic agent is a small molecule.

As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight of less than 5000 Daltons (5 kDa), preferably less than 3 kDa, still more preferably less than 2 kDa, and most preferably less than 1 kDa. In some cases it is preferred that a small molecule have a molecular weight equal to or less than 700 Daltons.

Exemplary therapeutic agents include, but are not limited to, those found in Harrison's Principles of Internal Medicine, 13^{th} Edition, Eds. T. R. Harrison et al. McGraw-Hill N.Y., NY; Physicians' Desk Reference, 50^{th} Edition, 1997, Oradell New Jersey, Medical Economics Co.; Pharmacological Basis of Therapeutics, 8^{th} Edition, Goodman and Gilman, 1990; United States Pharmacopeia, The National Formulary, USP XII NF XVII, 1990, the complete contents of all of which are incorporated herein by reference.

Therapeutic agents include the herein disclosed categories and specific examples. It is not intended that the category be limited by the specific examples. Those of ordinary skill in the art will recognize also numerous other compounds that fall within the categories and that are useful according to the present disclosure. Examples include a radiosensitizer, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifingal agent, a vaccine, a protein, or a nucleic acid. In a further aspect, the pharmaceutically active agent can be coumarin, albumin, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritic antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetaminophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists such as clonidine; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; antiarrhythmic agents such as quinidine, lidocaine, tocainide hydrochloride, mexiletine hydrochloride, digoxin, verapamil hydrochloride, propafenone hydrochloride, flecainide acetate, procainamide hydrochloride, moricizine hydrochloride, and disopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as Coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hdyrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides.

As noted above, any therapeutic agent can be encapsulated. In some embodiments, the therapeutic agent(s) for use in the present disclosure include, but are not limited to, those requiring relatively frequent dosing. For example, those used in the treatment of diabetes. In some embodiments, the therapeutic agent is an agent known in the art for treatment of diabetes. Exemplary therapeutic agents for treatment of diabetes, e.g., type 2 diabetes include, but are not limited to, Meglitinides, such as Repaglinide (Prandin) and Nateglinide (Starlix); Sulfonylureas, such as Glipizide (Glucotrol), Glimepiride (Amaryl), and Glyburide (DiaBeta, Glynase); Dipeptidy peptidase-4 (DPP-4) inhibitors, such as Saxagliptin (Onglyza), Sitagliptin (Januvia), and Linagliptin (Tradjenta); Biguanides, such as Metformin (Fortamet, Glucophage, others); Thiazolidinediones, such as Rosiglitazone (Avandia) and Pioglitazone (Actos); Alpha-glucosidase inhibitors, such as Acarbose (Precose) and Miglitol (Glyset); Amylin mimetics, such as Pramlintide (Symlin); and Incretin mimetics, such as Exenatide (Byetta) and Liraglutide (Victoza).

In some embodiments, therapeutic agent is a GLP-1 receptor agonist. A “GLP-1 receptor agonist” refers to compounds having GLP-1 receptor activity. The GLP-1 receptor agonist compounds can optionally be amidated. The term “exendin” includes naturally occurring (or synthetic versions of naturally occurring) exendin peptides that are found in the salivary secretions of the Gila monster. Exendins of particular interest include exendin-3 and exendin-4. The exendins, exendin analogs, and exendin agonists for use in the methods described herein may optionally be amidated, and may also be in an acid form, pharmaceutically acceptable salt form, or any other physiologically active form of the molecule. As used herein, the term “GLP-1 receptor agonist” include compounds that elicit a biological activity of an exendin reference peptide (e.g., exendin-4) or a GLP-1 reference peptide when evaluated by art-known measures such as receptor binding studies or in vivo blood glucose assays as described, e.g., by Hargrove et al, Regulatory Peptides, 141:113-119 (2007), content of which is incorporated herein by reference in its entirety.

Generally, GLP-1 receptor agonists can include peptides and small molecules, as known in the art. Exemplary GLP-1 receptor agonists have been described, such as those in Drucker, Endocrinology 144(12):5145-5148 (2003); EP 0708179; Hjorth et al, J. Biol. Chem. 269(48): 30121-30124 (1994); Siegel et al, Amer. Diabetes Assoc. 57 Scientific Sessions, Boston (1997); Hareter et al, Amer. Diabetes Assoc. 57th Scientific Sessions, Boston (1997); Adelhorst et al, J. Biol. Chem. 269(9): 6275-6278 (1994); Deacon et al, 16th International Diabetes Federation Congress Abstracts, Diabetologia Supplement (1997); Irwin et al, Proc. Natl. Acad. Sci. USA. 94: 7915-7920 (1997); Mosjov, Int. J Peptide Protein Res. 40: 333-343 (1992); Goke et al. Diabetic Medicine 13: 854-860 (1996). Publications also disclose Black Widow GLP-1 and Ser2 GLP-1. See Holz et al. Comparative Biochemistry and Physiology, Part B 121: 177-184 (1998) and Ritzel et al, “A synthetic glucagon-like peptide-1 analog with improved plasma stability,” J. Endocrinol. 159(1): 93-102 (1998), content of all of which is incorporated herein by reference in their entirety.

Exemplary GLP-1 receptor agonists include exenatide; liraglutide; lixisenatide; dulaglutide; albiglutide; taspoglutide; native exendins; exendin analogs; exendin-4; exendin-4 analogue; exendin agonists; native GLP-1; GLP-1(7-37); GLP-1(7-37) agonists; GLP-1(7-36)-amide; Arg³⁴, Lys²⁶(N^{E}—(Y-Glu(Nc′-hexadecanoyl)))-GLP-1(7-37); Gly⁸-GLP-1(7-36)amide; Gly⁸-GLP-1(7-37); Val⁸-GLP-1(7-36)-amide; Val⁸GLP-1(7-37); Val⁸Asp²²-GLP-1(7-36)-amide; Val⁸Asp²²GLP-1(7-37); Val⁸Glu²²-GLP-1(7-36)-amide; Val⁸Glu²²GLP-1(7-37); Val⁸Lys²²-GLP-1(7-36)-amide; Val⁸Lys²²GLP-1(7-37); Val⁸Arg²²-GLP-1(7-36)-amide; Val⁸Arg²²GLP-1(7-37); Val⁸His²²-GLP-1(7-36)-amide; Val⁸His²²GLP-1(7-37); or a functional analog or derivative thereof.

Additional GLP-1 receptor agonists include those described, for example, in US Patent App. Pub. No. 20050288248, No. 20060275288, No. 20090062192, No. 20100137212, No. 20100144621, No. 20110046071, No. 20110046071, No. 20110098217, No. 20110257092, No. 20110306549, No. 20120021972, No. 20120046222, and No. 20120148586; and U.S. Pat. No. 6,864,069, No. 6864069, No. 7041646, No. 7399745, No. 7488714, No. 7488715, No. 7488716, No. 7494978, No. 7833531, and No. 8178495, content of all of which is incorporated herein by reference in their entirety.

Analogs of GLP-1 are also useful as GLP-1 receptor agonists. Accordingly, in some embodiments, GLP-1 receptor agonists include without limitation those described in WO 98/43658 and WO 02/098348, and U.S. Pat. No. 5,512,549 and No. 7144863, content of all of which is incorporated herein by reference in their entirety.

In some embodiments, the therapeutic agent can be metformin (Glucophage, Glumetza), pioglitazone (Actos), glyburide (DiaBeta, Glynase), glipizide (Glucotrol and, Glucotrol XL), glimepiride (Amaryl), repaglinide (Prandin), nateglinide (Starlix), sitagliptin (Januvia), saxagliptin (Onglyza), exenatide (Byetta), liraglutide (Victoza), insulin lispro (Humalog), insulin aspart (NovoLog), insulin glargine (Lantus), insulin detemir (Levemir), and any combination thereof.

Generally, any amount of the therapeutic agent can be loaded into the silk matrix to provide a desired amount release over a period of time. For example, from about 0.1 ng to about 1000 mg of the therapeutic agent can be loaded in the silk matrix. In some embodiment, amount of therapeutic agent in the composition is selected from the range about from 0.001% (w/w) up to 95% (w/w), preferably, from about 5% (w/w) to about 75% (w/w), and most preferably from about 10% (w/w) to about 60% (w/w) of the total composition. In some embodiments, amount of amount of the therapeutic agent in the composition is from about 0.01% to about 95% (w/v), from about 0.1% to about 90% (w/v), from about 1% to about 85% (w/v), from about 5% to about 75% (w/v), from about 10% to about 65% (w/v), or from about 10% to about 50% (w/v), of the total composition.

In some embodiments, amount of the therapeutic agent in the composition is from about 0.01% to about 5% (w/v), from about 0.05% to about 4% (w/v), from about 0.1% to about 2.5% (w/v), from about 0.25% to about 2% (w/v), from about 0.3% to about 1.5% (w/v), from about 0.4% to about 1% (w/v) of the total composition. In some embodiments, amount of the therapeutic agent in the composition is from about 0.01% to about 5% (w/v), from about 0.02% to about 4% (w/v), from about 0.03% to about 3% (w/v), from about 0.04% to about 2% (w/v), from about 0.05% to about 1% (w/v), from about 0.055% to about 0.1% (w/v) of the total composition. In some embodiments, amount of the therapeutic agent in the composition is about 0.42% (w/v), about 0.06% (w/v), or about 0.12% (w/v) of the total composition.

In some embodiments, the silk-based drug-delivery composition described herein further comprises at least one biocompatible polymer, including at least two biocompatible polymers, at least three biocompatible polymers or more. Exemplary biocompatible polymers include, but are not limited to, a poly-lactic acid (PLA), poly-glycolic acid (PGA), poly-lactide-co-glycolide (PLGA), polyesters, poly(ortho ester), poly(phosphazine), poly(phosphate ester), polycaprolactone, gelatin, collagen, fibronectin, keratin, polyaspartic acid, alginate, chitosan, chitin, hyaluronic acid, pectin, polyhydroxyalkanoates, dextrans, and polyanhydrides, polyethylene oxide (PEO), poly(ethylene glycol) (PEG), triblock copolymers, polylysine, any derivatives thereof and any combinations thereof.

In some embodiments, the biocompatible polymer(s) can be integrated homogenously or heterogeneously within the bulk of the silk matrix. In other embodiments, the biocompatible polymer(s) can be coated on a surface of the silk matrix. In some embodiments, the biocompatible polymer(s) can be covalently or non-covalently linked to silk in the silk matrix. In some embodiments, the biocompatible polymer(s) can be blended with silk within the silk matrix.

In some embodiments, the biocompatible polymer is PEG or PEO. As used herein, the term “polyethylene glycol” or “PEG” means an ethylene glycol polymer that contains about 20 to about 2000000 linked monomers, typically about 50-1000 linked monomers, usually about 100-300. PEG is also known as polyethylene oxide (PEO) or polyoxyethylene (POE), depending on its molecular weight. Generally PEG, PEO, and POE are chemically synonymous, but historically PEG has tended to refer to oligomers and polymers with a molecular mass below 20,000 g/mol, PEO to polymers with a molecular mass above 20,000 g/mol, and POE to a polymer of any molecular mass. PEG and PEO are liquids or low-melting solids, depending on their molecular weights. PEGs are prepared by polymerization of ethylene oxide and are commercially available over a wide range of molecular weights from 300 g/mol to Ser. No. 10/000,000 g/mol. While PEG and PEO with different molecular weights find use in different applications, and have different physical properties (e.g. viscosity) due to chain length effects, their chemical properties are nearly identical. Different forms of PEG are also available, depending on the initiator used for the polymerization process—the most common initiator is a monofunctional methyl ether PEG, or methoxypoly(ethylene glycol), abbreviated mPEG. Lower-molecular-weight PEGs are also available as purer oligomers, referred to as monodisperse, uniform, or discrete PEGs are also available with different geometries.

As used herein, the term PEG is intended to be inclusive and not exclusive. The term PEG includes poly(ethylene glycol) in any of its forms, including alkoxy PEG, difunctional PEG, multiarmed PEG, forked PEG, branched PEG, pendent PEG (i.e., PEG or related polymers having one or more functional groups pendent to the polymer backbone), or PEG with degradable linkages therein. Further, the PEG backbone can be linear or branched. Branched polymer backbones are generally known in the art. Typically, a branched polymer has a central branch core moiety and a plurality of linear polymer chains linked to the central branch core. PEG is commonly used in branched forms that can be prepared by addition of ethylene oxide to various polyols, such as glycerol, pentaerythritol and sorbitol. The central branch moiety can also be derived from several amino acids, such as lysine. The branched poly(ethylene glycol) can be represented in general form as R(-PEG-OH)m in which R represents the core moiety, such as glycerol or pentaerythritol, and m represents the number of arms. Multiarmed PEG molecules, such as those described in U.S. Pat. No. 5,932,462, which is incorporated by reference herein in its entirety, can also be used as biocompatible polymers.

Some exemplary PEGs include, but are not limited to, PEG20, PEG30, PEG40, PEG60, PEG80, PEG100, PEG115, PEG200, PEG 300, PEG400, PEG500, PEG600, PEG1000, PEG1500, PEG2000, PEG3350, PEG4000, PEG4600, PEG5000, PEG6000, PEG8000, PEG11000, PEG12000, PEG15000, PEG 20000, PEG250000, PEG500000, PEG100000, PEG2000000 and the like. In some embodiments, PEG is of MW 10,000 Dalton. In some embodiments, PEG is of MW 100,000, i.e. PEO of MW 100,000.

The silk-based drug delivery composition can comprise any desired amount of PEG, PEO or POE. For example, the silk-based drug delivery composition can comprise from about 0.01% to about 50% of PEG, PEO or POE. Amount of PEG, PEO or POE can be based on weight, volume or moles of the total of the silk-based drug delivery composition. Thus, amount of PEG, PEO or POE present in the silk-based drug delivery composition can be weight/weight, weight/volume, volume/weight, or mole/mole. In some embodiments, amount of PEG, PEO or POE in the silk-based drug delivery composition can range from about 0.1% to about 25% (w/v), from about 0.25% to about 20% (w/v), from about 0.5% to about 15% (w/v), from about 0.75% to about 10% (w/v), from about 1% to about 9% (w/v), from about 2% to about 7% (w/v), from about 3% to about 6% (w/v), or from about 4.5 to about 5.5% (w/v). In some embodiments, amount of PEG, PEO or POE in the silk-based drug delivery composition is from about 0 to about 10% (w/v). In some embodiments, amount of PEG, PEO or POE in the silk-based drug delivery composition is from about 0.1 to about 7.5% (w/v). In some embodiments, amount of PEG, PEO or POE in the silk-based drug delivery composition is about 0.25% (w/v), about 1% (w/v) or about 5% (w/v).

The inventors have discovered inter alia that presence of albumin in the silk-based drug delivery compositions described herein can alter the release kinetics of the therapeutic agent from the gel. Without wishing to be bound by a theory, presence of albumin in the silk-based drug delivery composition can provide a diffusion barrier to regulate the release of the therapeutic agent from the composition. Thus, in some embodiments, the silk-based drug-delivery composition described herein further comprises albumin.

Albumin is a simple protein found in serum and has a molecular weight of about 66,000 Daltons. Albumin is produced in the liver and is the most abundant blood plasma protein. Albumin polypeptides are important in regulating blood volume by maintaining appropriate colloid osmotic pressure. Human serum albumin is a monomer of 585 amino acid residues, and includes three homologous a-helical domains: domain I, domain II and domain III. Each domain contains 10 helices and is divided into antiparallel six-helix and four-helix subdomains. Deletion studies suggest that domain III alone is sufficient for binding to FcRn (Chaudhury et al., Biochemistry 2006, 45:4983-4990). A truncated human albumin that does not bind FcRn and has a low serum level has been identified (Andersen et al., Clin Biochem., 2010, 43(45):367-72. Epub 2009 Dec. 16).

Albumin is known to bind and carry a Wide variety of small molecules, including lipid soluble hormones, bile salts, unconjugated bilirubin, fatty acids, calcium, ions, transferrin, hemin, and tryptophan. Albumin also binds various drugs such as Warfarin, phenobutazone, clofibrate and phenytoin, and its binding can alter the drugs' pharrnacokinetic properties.

The albumin can be a naturally occurring albumin, an albumin related protein or a variant thereof such as a natural or engineered variant. Variants include polymorphisms, fragments such as domains and subdomains, fragments and/or fusion proteins. An albumin can comprise the sequence of an albumin protein obtained from any source. Typically the source is mammalian such as human or bovine. In some embodiments, the serum albumin is human serum albumin (“HSA”). The term “human serum albumin” includes a serum albumin having an amino acid sequence naturally occurring in humans, and variants thereof. The HSA coding sequence is obtainable by known methods for isolating cDNA corresponding to human genes, and is also disclosed in, for example, EP 0 073 646 and EP 0 286 424, content of both of which is incorporated by reference in their entirety. A fragment or variant can be functional or non-functional. For example, a fragment or variant can retain the ability to bind to an albumin receptor such as FcRn to at least 10, 20, 30, 40, 50, 60, 70, 80, 90 or 100% of the ability of the parent albumin (from which the fragment or variant derives) to bind to the receptor. Relative binding ability can be determined by methods known in the art such as surface plasmon resonance studies.

The albumin can be a naturally-occurring polymorphic variant of human albumin or of a human albumin analogue. Generally, variants or fragments of human albumin will have at least 5%, 10%, 15%, 20%, 30%, 40%, 50%, 60%, 70%, (preferably at least 80%, 90%, 95%, 100%, 105% or more) of human albumin's ligand binding activity (for example FcRN-binding), mole for mole.

The albumin can comprise the sequence of bovine serum albumin. The term “bovine serum albumin” includes a serum albumin having an amino acid sequence naturally occurring in cows, for example as taken from Swissprot accession number P02769, and variants thereof as defined herein. The term “bovine serum albumin” also includes fragments of full-length bovine serum albumin or variants thereof, as defined herein.

A number of proteins are known to exist within the albumin family. Accordingly, the albumin can comprise the sequence of an albumin derived from one of serum albumin from African clawed frog (e.g., see Swissprot accession number P08759-1), bovine (e.g., see Swissprot accession number P02769-1), cat (e.g., see Swissprot accession number P49064-1), chicken (e.g., see Swissprot accession number P19121-1), chicken ovalbumin (e.g., see Swissprot accession number P01012-1), cobra ALB (e.g., see Swissprot accession number Q91134-1), dog (e.g., see Swissprot accession number P49822-1), donkey (e.g., see Swissprot accession number QSXLE4-1), European water frog (e.g., see Swissprot accession number Q9YGH6-1), blood fluke (e.g., see Swissprot accession number AAL08579 and Q95VB7-1), Mongolian gerbil (e.g., see Swissprot accession number 035090-1 and JC5838), goat (e.g., see Swissprot accession number B3VHM9-1 and as available from Sigma as product no. A2514 or A4164), guinea pig (e.g., see Swissprot accession number Q6WDN9-1), hamster (see DeMarco et al. (2007). International Journal for Parasitology 37(11): 1201-1208), horse (e.g., see Swissprot accession number P35747-1), human (e.g., see Swissprot accession number P02768-1), Australian Lung-fish (e.g., see Swissprot accession number P83517)-1 22.8 101 fish), macaque (Rhesus monkey) (e.g., see Swissprot accession number Q28522-), mouse (e.g., see Swissprot accession number P07724-1), North American bull frog (e.g., see Swissprot accession number P21847-1), pig (e.g., see Swissprot accession number P08835-1), pigeon (e.g. as defined by Khan et al, 2002,1112. J. Biol. Macromol, 30β-4), 171-8), rabbit (e.g., see Swissprot accession number P490 65-1), rat (e.g., see Swissprot accession number P02770-1), salamander (e.g., see Swissprot accession number Q8UW05-1), salmon ALB1 (e.g., see Swissprot accession number P21848-1), salmon ALB2 (e.g., see Swissprot accession number Q03156-1), sea lamprey (e.g., see Swissprot accession number Q91274-1 and 042279-1) sheep (e.g., see Swissprot accession number P14639-1), Sumatran orangutan (e.g., see Swissprot accession number Q5NVH5-1), tuatara (e.g., see Swissprot accession number Q8JIA9-1), turkey ovalbumin (e.g., see Swissprot accession number 073860-1), Western clawed frog (e.g., see Swissprot accession number Q6D.I95-1), and includes variants and fragments thereof as defined herein.

Many naturally occurring mutant forms of albumin are known. Many are described in Peters, (1996, All About Albumin: Biochemistry, Genetics and Medical Applications, Academic Press, Inc., San Diego, Calif., p. 170-181), content of which is incorporated herein by reference. A variant as defined herein can be one of these naturally occurring mutants such as those described in Minchiotti et al. (2008). Hum Mutat 29(8): 1007-16, content of which is incorporated herein by reference in its entirety.

A “variant albumin” refers to an albumin protein wherein at one or more positions there have been amino acid insertions, deletions, or substitutions, either conservative or non-conservative, provided that such changes result in an albumin protein for which at least one basic property, for example binding activity (type of and specific activity e.g. binding to bilirubin or a fatty acid such as a long-chain fatty acids, for exampleoleic (C18:1), palmitic (C16:0), linoleic (C18:2), stearic (C18:0), arachidonic (C20:4) and/or palmitoleic (C16:1)), osmolarity (oncotic pressure, colloid osmotic pressure), behaviour in a certain pH-range (pH-stability) has not significantly been changed. “Significantly” in this context means that one skilled in the art would say that the properties of the variant can still be different but would not be unobvious over the ones of the original protein, e.g. the protein from which the variant is derived. Such characteristics can be used as additional selection criteria in the invention.

The term albumin also encompasses albumin variants, such as genetically engineered forms, mutated forms, and fragments etc. having one or more binding sites that are analogous to a binding site unique for one or more albumins as defined above. By analogous binding sites in the context of the invention are contemplated structures that are able to compete with each other for binding to one and the same ligand structure.

In some embodiments, the albumin can be human serum albumin extracted from serum or plasma, or recombinant human albumin (rHA) produced by transforming or transfecting an organism with a nucleotide coding sequence encoding the amino acid sequence of human serum albumin, including rHA produced using transgenic animals or plants.

In one embodiment, albumin is bovine serum albumin, includes variants and fragments thereof.

The silk-based drug delivery composition can comprise any desired amount of albumin. For example, the silk-based drug delivery composition can comprise from about 0.1% to about 50% of albumin. Amount of albumin can be based on weight, volume or moles of the total of the silk-based drug delivery composition. Thus, amount of albumin present in the silk-based drug delivery composition can be weight/weight, weight/volume, volume/weight, or mole/mole. In some embodiments, amount of albumin in the silk-based drug delivery composition can range from about 0.5% to about 25% (w/v), from about 1% to about 20% (w/v), from about 2% to about 15% (w/v), from about 3% to about 10% (w/v), from about 4% to about 8% (w/v), from about 5% to about 7% (w/v).. In some embodiments, amount of albumin in the silk-based drug delivery composition can range from 0 to about 20% (w/v). In one embodiment, amount of albumin in the silk-based drug delivery composition is about 5% (w/v).

In some embodiments, the albumin can be integrated homogenously or heterogeneously within the bulk of the silk matrix. In other embodiments, the albumin can be coated on a surface of the silk matrix. In some embodiments, the albumin can be covalently or non-covalently linked to silk in the silk matrix. In some embodiments, the albumin can be blended with silk within the silk matrix.

In some embodiments, the silk-based drug delivery composition can further comprise additives. Some exemplary additives include biologically or pharmaceutically active compounds. Examples of biologically active compounds include, but are not limited to: cell attachment mediators, such as collagen, elastin, fibronectin, vitronectin, laminin, proteoglycans, or peptides containing known integrin binding domains e.g. “RGD” integrin binding sequence, or variations thereof, that are known to affect cellular attachment (Schaffner P & Dard 2003 Cell Mol Life Sci. January; 60(1):119-32; Hersel U. et al. 2003 Biomaterials. November; 24(24):4385-415); biologically active ligands; and substances that enhance or exclude particular varieties of cellular or tissue ingrowth. Other examples of additive agents that enhance proliferation or differentiation include, but are not limited to, osteoinductive substances, such as bone morphogenic proteins (BMP); cytokines, growth factors such as epidermal growth factor (EGF), platelet-derived growth factor (PDGF), insulin-like growth factor (IGF-I and II) TGF-β1. As used herein, the term additive also encompasses antibodies, DNA, RNA, modified RNA/protein composites, glycogens or other sugars, and alcohols.

The inventors have discovered inter alia that the therapeutic agent is released in a sustained release manner from the silk-based drug delivery compositions described herein. In other words, the silk-based drug delivery composition described herein is a sustained delivery composition. As used herein, the term “sustained delivery” refers to continual delivery of a therapeutic agent in vivo or in vitro over a period of time following administration. For example, sustained release can occur over a period of at least about 3 days, at least about a week, at least about two weeks, at least about three weeks, at least about four weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months or longer. In some embodiments, the sustained release can occur over a period of more than one month or longer. In some embodiments, the sustained release can occur over a period of at least about three months or longer. In some embodiments, the sustained release can occur over a period of at least about six months or longer. In some embodiments, the sustained release can occur over a period of at least about nine months or longer. In some embodiments, the sustained release can occur over a period of at least about twelve months or longer.

Sustained delivery of the therapeutic agent in vivo can be demonstrated by, for example, the continued therapeutic effect of the agent over time. Alternatively, sustained delivery of the therapeutic agent can be demonstrated by detecting the presence or level of the therapeutic agent or a metabolite thereof in vivo over time. By way of example only, sustained delivery of the therapeutic agent, upon administration, can be detected by measuring the amount of therapeutic agent or a metabolite thereof present in blood serum, a tissue or an organ of a subject.

The release rate of a therapeutic agent from the silk-based drug delivery composition can be adjusted by a number of factors such as silk matrix composition and/or concentration, porous property of the silk matrix, molecular size of the therapeutic agent, and/or interaction of the therapeutic agent with the silk matrix. For example, if the therapeutic agent has a higher affinity with the silk matrix, the release rate is usually slower than the one with a lower affinity with the silk matrix. Additionally, when a silk matrix has larger pores, the encapsulated therapeutic agent is generally released from the silk matrix faster than from a silk matrix with smaller pores.

The release profiles of the therapeutic agent from the silk matrix can be modulated by a number of factors such as amounts and/or molecular size of the therapeutic agents loaded in the silk matrix, porosity of the silk matrix, amounts of silk fibroin in the silk matrix and/or contents of beta-sheet conformation structures in a silk matrix, binding affinity of the therapeutic agent to a silk matrix, and any combinations thereof.

The silk-based drug delivery composition can provide or release an amount of the therapeutic agent, which provides a therapeutic effect similar to as provided by a recommended dosage of the therapeutic agent for the same period of time. For example, if the recommended dosage for the therapeutic agent is once daily, then the silk-based drug delivery composition releases that amount of therapeutic agent, which is sufficient to provide a similar therapeutic effect as provided by the once daily dosage.

Daily release of the therapeutic agent can range from about 1 ng/day to about 1000 mg/day. For example, amount released can be in a range with a lower limit of from 1 to 1000 (e.g., every integer from 1 to 1000) and upper limit of from 1 to 1000 (e.g. every integer from 1 to 1000), wherein the lower and upper limit units can be selected independently from ng/day, μg/day, mg/day, or any combinations thereof.

In some embodiments, daily release can be from about 1 μg/day to about 10 mg/day, from about 0.25 μg/day to about 2.5 mg/day, or from about 0.5 μg/day to about 5 mg/day. In some embodiments, daily release of the therapeutic agent can range from about 100 ng/day to 1 mg/day, for example, or about 500 ng/day to 5 mg/day, or about 100 μg/day. In some embodiments, daily release of the therapeutic agent is from about 5 to about 60 μg/day. In some embodiments, daily release of the therapeutic agent is about 10 μg/day.

The inventors have discovered that release of the therapeutic agent from the silk reservoir implant or silk injectable reservoir composition follows near zero-order release kinetics over a period of time. For example, near zero-order release kinetics can be achieved over a period of one week, two weeks, three weeks, four weeks, one month, two months, three months, four months, five months, six months, twelve months, one year or longer.

In some embodiments, no significant apparent initial burst release is observed from the drug delivery composition described herein. Accordingly, in some embodiments, the initial burst of the therapeutic agent within the first 48, 24, 18, 12, or 6 hours of administration is less than 25%, less than 20%, less than 15%, less than 10%, less than 9%, less than 8%, less than 7%, less than 6%, less than 5%, less than 4%, less than 3%, less than 2%, or less than 1% of the total amount of therapeutic agent loaded in the drug delivery composition. In some embodiments, there is no initial burst of therapeutic agent within the first 6 or 12 hours, 1, 2, 3, 4, 5, 6, 7 days, 1 and 2 weeks of administration.

The silk-based drug delivery composition can stabilize the activity, e.g., bioactivity, of a therapeutic agent under a certain condition, e.g., under an in vivo physiological condition. See, for example, U.S. Provisional Application No. 61/477,737, filed Apr. 21, 2011 and International Patent Application No. PCT/US2012/034643, filed Apr. 23, 2012, the content of both of which is incorporated herein by reference in its entirety. Accordingly, the silk-based drug delivery composition can increase the in vivo half-life of the therapeutic agent. For example, in vivo half-life of an encapsulated therapeutic agent can increase by at least 5%, at least 10%, at least 15%, at least 20%, at least 25%, at least 30%, at least 35%, at least 40%, at least 50%, at least 60%, at least 70%, at least 80%, at least 90%, at least 1-fold, at least 1.5-fold, at least 2-fold, at least 5-fold, at least 5-fold, at least 10-fold or more relative to the non-encapsulated therapeutic agent. In some embodiments, in vivo half-life of the encapsulated therapeutic agent is at least 5%, at least 10%, at least 15%, at least 20%, at least 25%, at least 30%, at least 35%, at least 40%, at least 50%, at least 60%, at least 70%, at least 80%, at least 90%, at least 1-fold, at least 1.5-fold, at least 2-fold, at least 5-fold, at least 5-fold, at least 10-fold or longer than the in vivo half-life of the therapeutic agent when not encapsulated in the silk matrix.

Without wishing to be bound by theory, the silk-based drug delivery composition can provide a longer therapeutic effect. Stated another way, an increase in in vivo half-life of a therapeutic agent can allow loading of a smaller amount of the therapeutic agent for the same duration of therapeutic effect. Accordingly, encapsulating a therapeutic agent in a silk matrix can increase the duration of effect for the therapeutic agent. For example, amount of therapeutic agent encapsulated in the silk-based drug delivery composition provides a therapeutic effect for a period of time, which is longer than when the same amount of therapeutic agent is administered without the silk-based drug delivery composition. In some embodiments, duration of therapeutic effect is at least one day, at least two days, at least three days, at least four days, at least five days, at least six days, at least seven days, at least one week, at least two weeks, at least three weeks, at least four weeks, at least one month, at least two months, at least three months, at least four months, at least five months, at least six months or longer than the duration of effect when the therapeutic agent is administered without the silk-based drug delivery composition.

In some embodiments, the duration of therapeutic effect from a single dosage is at least one day, at least two days, at least three days, at least four days, at least five days, at least six days, at least seven days, at least one week, at least two weeks, at least three weeks, at least four weeks, at least one month, at least two months, at least three months, at least four months, at least five months, at least six months or longer.

Accordingly, the silk-based drug delivery compositions described herein can comprise the therapeutic agent in an amount which is less than the amount recommended for one dosage of the therapeutic agent. For example, if the recommended dosage of the therapeutic agent is X amount then the silk matrix can comprise a therapeutic agent in an amount of about 0.9×, about 0.8×, about 0.7×, about 0.6×, about 0.5×, about 0.4×, about 0.3×, about 0.2×, about 0.1× or less. Without wishing to be bound by a theory, this can allow administering a lower dosage of the therapeutic agent in a silk matrix to obtain a therapeutic effect which is similar to when a higher dosage is administered without the silk matrix.

In some embodiments, amount of the therapeutic agent dispersed or encapsulated in the silk matrix can be more than the amount generally recommended for one dosage of the same therapeutic agent administered for a particular indication. For example, if the recommended dosage of the therapeutic agent is X amount then the silk matrix can encapsulate a therapeutic agent in an amount of about 1.25×, about 1.5×, about 1.75×, about 2×, about 2.5×, about 3×, about 4×, about 5×, about 6×, about 7×, about 8×, about 9×, about 10× or more. Without wishing to be bound by a theory, this can allow administering the therapeutic agent in a silk matrix to obtain a therapeutic effect which is similar to one obtained with multiple administration of the therapeutic agent administered without the silk matrix described herein.

In some embodiments, the amount of the therapeutic agent encapsulated in the silk matrix can be essentially the same amount recommended for one dosage of the therapeutic agent. For example, if the recommended dosage of the therapeutic agent is X amount, then the silk-based composition can comprise about X amount of the therapeutic agent. Since the silk-based drug delivery compositions described herein can increase the duration of effect for the therapeutic agent, this can allow less frequent administration of the therapeutic agent to obtain a therapeutic effect over a longer period of time.

Furthermore, the silk-based drug delivery composition can increase bioavailability of the encapsulated therapeutic agent. As used herein, the term “bioavailability” refers to the amount of a substance available at a given site of physiological activity after administration. Bioavailability of a given substance is affected by a number of factors including but not limited to degradation and absorption of that substance. Administered substances are subject to excretion prior to complete absorption, thereby decreasing bioavailability. In some embodiments, bioavailability of an encapsulated therapeutic agent can increase by at least 5%, at least 10%, at least 15%, at least 20%, at least 25%, at least 30%, at least 35%, at least 40%, at least 50%, at least 60%, at least 70%, at least 80%, at least 90%, at least 1-fold, at least 1.5-fold, at least 2-fold, at least 5-fold, at least 5-fold, at least 10-fold or more relative to the non-encapsulated therapeutic agent.

Without wishing to be bound by a theory, silk-based drug delivery compositions can allow the frequency of administration of the therapeutic agent to be reduced by a factor of F=(Y2−Y1)/Y2, wherein Y1 is the duration of the therapeutic effect produced by the current dosage of the therapeutic agent without silk matrix recommended for a particular indication, and Y2 is the duration of the therapeutic effect produced by the same amount of the therapeutic agent present in a silk-based drug delivery composition described herein. Frequency of administration for the silk matrix encapsulated therapeutic agent can be calculated using the formula:

Frequency of administration=Z×F [1]

wherein Z is number of administrations over a given period of time.

For example, if the duration of the therapeutic effect produced by the current dosage of the therapeutic agent without silk matrix recommended for a particular indication is one month (Y1=1 month) and the duration of the therapeutic effect produced by the same amount of the therapeutic agent present in a silk-based drug delivery composition described herein is two month, then the frequency of administration is reduced by a factor of ½ (e.g., Y2=2 months, and Y1=1 month). The frequency of administration is reduced to about once every two months. That is, instead of having an administration of the therapeutic agent once a month with the current administration protocol, the methods and/or compositions of the invention can reduce frequency of administration to about once every two months. Similarly, if the frequency of administration is reduced by a factor of ⅔ (e.g., Y2=3 months, and Y1=1 month), the methods and/or compositions described herein can reduce frequency of administration to about once every 3 months.

In some embodiments, the frequency of administration of the therapeutic agent can be reduced by a factor of at least about 1/500, at least about 1/250, at least about 1/225, at least about 1/200, at least about 1/175, at least about 1/150, at least about 1/125, at least about 1/100, at least about 1/90, at least about 1/80, at least about 1/70, at least about 1/60, at least about 1/50, at least about 1/30, at least about 1/25, at least about 1/20, at least about 1/19, at least about 1/18, at least about 1/17, at least about 1/16, at least about 1/15, at least about 1/14, at least about 1/13, at least about 1/12, at least about 1/11, at least about 1/10, at least about 1/9, at least about ⅛, at least about 1/7, at least about ⅙, at least about ⅕, at least about ¼, at least about ⅓, at least about ½, at least about 1/1.75, at least about 1/1.5, at least about 1/1.25, at least about 1/1.1, or more.

In yet another aspect, provided herein is a method for sustained delivery in vivo of a therapeutic agent. The method comprising administering a silk-based drug delivery composition described herein to a subject. Without wishing to be bound by a theory, the therapeutic agent can be released in a therapeutically effective amount daily.

As used herein, the term “therapeutically effective amount” means an amount of the therapeutic agent which is effective to provide a desired outcome. Determination of a therapeutically effective amount is well within the capability of those skilled in the art. Generally, a therapeutically effective amount can vary with the subject's history, age, condition, sex, as well as the severity and type of the medical condition in the subject, and administration of other agents that inhibit pathological processes in neurodegenerative disorders.

Furthermore, therapeutically effective amounts will vary, as recognized by those skilled in the art, depending on the specific disease treated, the route of administration, the excipient selected, and the possibility of combination therapy. In some embodiments, the therapeutically effective amount can be in a range between the ED50 and LD50 (a dose of a therapeutic agent at which about 50% of subjects taking it are killed). In some embodiments, the therapeutically effective amount can be in a range between the ED50 (a dose of a therapeutic agent at which a therapeutic effect is detected in at least about 50% of subjects taking it) and the TD50 (a dose at which toxicity occurs at about 50% of the cases). In some embodiments, the therapeutically effective amount can be an amount determined based on the current dosage regimen of the same therapeutic agent administered in a non-silk matrix. For example, an upper limit of the therapeutically effective amount can be determined by a concentration or an amount of the therapeutic agent delivered or released on the day of administration with the current dosage of the therapeutic agent in a non-silk matrix; while the lower limit of the therapeutically effective amount can be determined by a concentration or an amount of the therapeutic agent on the day at which a fresh dosage of the therapeutic agent in a non-silk matrix is required. Guidance regarding the efficacy and dosage which will deliver a therapeutically effective amount of a compound can be obtained from animal models of condition to be treated.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the IC₅₀ (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay. Examples of suitable bioassays include DNA replication assays, transcription based assays, and immunological assays.

The dosage can be determined by a physician and adjusted, as necessary, to suit observed effects of the treatment. Generally, the therapeutic agents are administered so that the therapeutic agent is given at a dose from 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. For protein therapeutic agents, one preferred dosage is 0.1 mg/kg of body weight (generally 10 mg/kg to 20 mg/kg).

As disclosed herein, the silk-based drug delivery can provide a therapeutically effective amount of the therapeutic agent to a subject for a period of time which is similar to or longer than the period of time when the therapeutic agent is administered without the silk-based drug delivery composition. For example, amount of therapeutic agent released over a day provides a similar therapeutic effect as provided by the recommended daily dosage of the therapeutic agent when administered without the silk-based drug delivery composition.

For administration to a subject, the silk-based drug delivery composition can be formulated in pharmaceutically acceptable compositions which comprise a drug delivery composition, formulated together with one or more pharmaceutically acceptable carriers (additives) and/or diluents. The drug delivery composition can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery composition. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alchols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

Pharmaceutically-acceptable antioxidants include, but are not limited to, (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lectithin, propyl gallate, alpha-tocopherol, and the like; and (3) metal chelating agents, such as citric acid, ethylenediamine tetraacetic acid (EDTA), sorbitol, tartaric acid, phosphoric acids, and the like.

As used herein, the term “administered” refers to the placement of a drug delivery composition into a subject by a method or route which results in at least partial localization of the pharmaceutically active agent at a desired site. A drug delivery composition described herein can be administered by any appropriate route which results in effective treatment in the subject, i.e., administration results in delivery to a desired location in the subject where at least a portion of the pharmaceutically active agent is delivered. Exemplary modes of administration include, but are not limited to, implant, injection, infusion, instillation, implantation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

In some embodiments, a drug delivery composition described herein can be implanted in a subject. As used herein, the term “implanted,” and grammatically related terms, refers to the positioning of the silk-based drug delivery composition in a particular locus in the subject, either temporarily, semi-permanently, or permanently. The term does not require a permanent fixation of the silk-based drug delivery composition in a particular position or location. Exemplary in vivo loci include, but are not limited to site of a wound, trauma or disease.

In some embodiments, the silk-based drug delivery compositions described herein are suitable for in vivo delivery to a subject by an injectable route. One delivery route is injectable, which includes intravenous, intramuscular, subcutaneous, intraperitoneal, intrathecal, epidural, intra-arterial, intra-articular and the like. Other delivery routes, such as topical, oral, rectal, nasal, pulmonary, vaginal, buccal, sublingual, transdermal, transmucosal, otic or intraocular, could also be practiced.

Accordingly, in some embodiments, the composition is in form of an injectable composition. As used herein, the term “injectable composition” generally refers to a composition that can be delivered or administered into a tissue with a minimally invasive procedure. The term “minimally invasive procedure” refers to a procedure that is carried out by entering a subject's body through the skin or through a body cavity or an anatomical opening, but with the smallest damage possible (e.g., a small incision, injection). In some embodiments, the injectable composition can be administered or delivered into a tissue by injection. In some embodiments, the injectable composition can be delivered into a tissue through a small incision on the skin followed by insertion of a needle, a cannula, and/or tubing, e.g., a catheter. Without wishing to be limited, the injectable composition can be administered or placed into a tissue by surgery, e.g., implantation. Some exemplary injectable compositions include, but are not limited to, solutions, hydrogels, gel-like particles, and/or microspheres.

To be clear, term “injectable” as in an “injectable formulation” and “injectables” refers to physical properties of a solution (e.g., formulation) suitable for administration by injection, such that there is a sufficient flow of the solution to pass through a needle or any other suitable means, and that such flow is generated with reasonable ease by a user. Syringes are commonly employed for delivering injections to subjects. In some embodiments, the injectable formulation can be provided as pre-filled syringes. In some embodiments, the injectable formulation can be provided as a ready-to-use formulation. In some embodiments, the injectable formulation can be provided as a kit.

In some embodiments, the injectable compositions can further comprise a pharmaceutically acceptable carrier. The compositions suitable for injection include sterile aqueous solutions or dispersions. The carrier can be a solvent or dispersing medium containing, for example, water, cell culture medium, buffers (e.g., phosphate buffered saline), polyol (for example, glycerol, propylene glycol, liquid polyethylene glycol, and the like), suitable mixtures thereof. In some embodiments, the pharmaceutical carrier can be a buffered solution (e.g. PBS).

Additionally, various additives which enhance the stability, sterility, and isotonicity of the injectable compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it may be desirable to include isotonic agents, for example, sugars, sodium chloride, and the like. The injectable compositions can also contain auxiliary substances such as wetting or emulsifying agents, pH buffering agents, gelling or viscosity enhancing additives, preservatives, colors, and the like, depending upon the preparation desired.

Viscosity of the injectable compositions can be modulated by controlling the weight percentage of silk fibroin fragments having a molecular weight sub-range (i) to (xviii). In some embodiments, the viscosity of the injectable composition can be further maintained at the selected level using a pharmaceutically acceptable thickening agent. In one embodiment, methylcellulose can be used because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected, and the desired viscosity for injection. The important point is to use an amount which will achieve the selected viscosity, e.g., addition of such thickening agents into some embodiments of the injectable compositions.

For injection, silk-based drug delivery compositions can be aspirated into a syringe and injected through a needle of gauge of about 10 to about 34 or about 18 to about 30. An exemplary delivery route is injection with a fine needle, which includes subcutaneous, ocular and the like. By fine needle is meant needles of at least 10 Gauge size, typically between about 18 Gauge and about 30 Gauge and above. In some embodiments, the fine needles can be at least as fine as 10 Gauge, at least as fine as 12 Gauge, at least as fine as 14 Gauge, at least as fine as 16 Gauge, at least as fine as 18 Gauge, at least as fine as 21 Gauge, at least as fine as 22 Gauge, at least as fine as 23 Gauge, at least as fine as 24 Gauge, at least as fine as 25 Gauge, at least as fine as 26 gauge, or at least as fine as 28 Gauge.

Without limitations, silk-based drug delivery compositions described herein can be used for administering, to a subject, a pharmaceutical agent that requires relatively frequent administration. For example, a pharmaceutically active agent that requires administration at least once every three months, at least once every two months, at least once every week, at least once daily for a period of time, for example over a period of at least one week, at least two weeks, at least three weeks, at least four weeks, at least one month, at least two months, at least three months, at least four months, at least five months, at least six months, at least one years, at least two years or longer.

As is known in the art, many therapeutic agents for treatment of chronic disorders or conditions require relatively frequent dosing. Thus, provided herein is method for treatment of a chronic disease or disorder in subject. The method comprises administering a silk-based drug delivery composition described herein or a pharmaceutical composition comprising a silk-based drug delivery composition described herein to subject in need thereof. The silk-based drug delivery comprises a therapeutic agent that requires frequent administration for treatment of chronic disease or condition under consideration.

Exemplary chronic diseases include, but are not limited to, anemia, autoimmune diseases including autoimmune vasculitis, cartilage damage, CIDP, Cystic Fibrosis, diabetes (e.g., insulin diabetes), graft vs. host disease, Hemophilia, infection or other disease processes, inflammatory arthritis, inflammatory bowel disease, inflammatory conditions resulting from strain, inflammatory joint disease, Lupus, lupus, Multiple Sclerosis, Myasthenia Gravis, Myositis, orthopedic surgery, osteoarthritis, Parkinson's Disease, psioriatic arthritis, rheumatoid arthritis, Sickle Cell Anemia, sprain, transplant rejection, trauma, and the like.

By “treatment, prevention or amelioration” is meant delaying or preventing the onset of such a disorder or reversing, alleviating, ameliorating, inhibiting, slowing down or stopping the progression, aggravation or deterioration the progression or severity of such a condition. In some embodiments, at least one symptom is alleviated by at least 20%, at least 30%, at least 40%, at least 50%, at least 60%, at least 70%, at least 80%, at least 90%, or at least 95% but not 100%, i.e. not a complete alleviation. In some embodiments, at least one symptom is completely alleviated.

In some embodiments, subject is need of treatment for diabetes. The terms “diabetes” and “diabetes mellitus” are used interchangeably herein. The World Health Organization defines the diagnostic value of fasting plasma glucose concentration to 7.0 mmol/l (126 mg/dl) and above for Diabetes Mellitus (whole blood 6.1 mmol/l or 110 mg/dl), or 2-hour glucose level ≧11.1 mmol/L (≧200 mg/dL). Other values suggestive of or indicating high risk for Diabetes Mellitus include elevated arterial pressure 140/90 mm Hg; elevated plasma triglycerides (≧1.7 mmol/L; 150 mg/dL) and/or low HDL-cholesterol (<0.9 mmol/L, 35 mg/dl for men; <1.0 mmol/L, 39 mg/dL women); central obesity (males: waist to hip ratio >0.90; females: waist to hip ratio >0.85) and/or body mass index exceeding 30 kg/m²; microalbuminuria, where the urinary albumin excretion rate 20 μg/min or albumin:creatinine ratio 30 mg/g).

A “pre-diabetic condition” refers to a metabolic state that is intermediate between normal glucose homeostasis, metabolism, and states seen in Diabetes Mellitus. Pre-diabetic conditions include, without limitation, Metabolic Syndrome (“Syndrome X”), Impaired Glucose Tolerance (IGT), and Impaired Fasting Glycemia (IFG). IGT refers to post-prandial abnormalities of glucose regulation, while IFG refers to abnormalities that are measured in a fasting state. The World Health Organization defines values for IFG as a fasting plasma glucose concentration of 6.1 mmol/L (100 mg/dL) or greater (whole blood 5.6 mmol/L; 100 mg/dL), but less than 7.0 mmol/L (126 mg/dL)(whole blood 6.1 mmol/L; 110 mg/dL). Metabolic Syndrome according to National Cholesterol Education Program (NCEP) criteria are defined as having at least three of the following: blood pressure ≧130/85 mm Hg; fasting plasma glucose ≧6.1 mmol/L; waist circumference >102 cm (men) or >88 cm (women); triglycerides ≧1.7 mmol/L; and HDL cholesterol <1.0 mmol/L (men) or 1.3 mmol/L (women).

“Impaired glucose tolerance” (IGT) is defined as having a blood glucose level that is higher than normal, but not high enough to be classified as Diabetes Mellitus. A subject with IGT will have two-hour glucose levels of 140 to 199 mg/dL (7.8 to 11.0 mmol) on the 75 g oral glucose tolerance test. These glucose levels are above normal but below the level that is diagnostic for Diabetes. Subjects with impaired glucose tolerance or impaired fasting glucose have a significant risk of developing Diabetes and thus are an important target group for primary prevention.

“Normal glucose levels” is used interchangeably with the term “normoglycemic” and refers to a fasting venous plasma glucose concentration of less than 6.1 mmol/L (110 mg/dL). Although this amount is arbitrary, such values have been observed in subjects with proven normal glucose tolerance, although some may have IGT as measured by oral glucose tolerance test (OGTT). A baseline value, index value, or reference value in the context of the present invention and defined herein can comprise, for example, “normal glucose levels.”

In general, treatment of Diabetes is determined by standard medical methods. A goal of Diabetes treatment is to bring sugar levels down to as close to normal as is safely possible. Commonly set goals are 80-120 milligrams per deciliter (mg/dl) before meals and 100-140 mg/dl at bedtime. A particular physician may set different targets for the patent, depending on other factors, such as how often the patient has low blood sugar reactions. Useful medical tests include tests on the patient's blood and urine to determine blood sugar level, tests for glycosylated hemoglobin level (HbAlc; a measure of average blood glucose levels over the past 2-3 months, normal range being 4-6%), tests for cholesterol and fat levels, and tests for urine protein level. Such tests are standard tests known to those of skill in the art (see, for example, American Diabetes Association, 1998). A successful treatment program can also be determined by having fewer patients in the program with complications relating to Diabetes, such as diseases of the eye, kidney disease, or nerve disease.

There are two general forms of Diabetes mellitus: (1) insulin dependent or Type 1 Diabetes (a.k.a., Juvenile Diabetes, Brittle Diabetes, Insulin Dependent Diabetes Mellitus (IDDM)) and (2) non-insulin-dependent or Type II Diabetes (a.k.a., NIDDM). Type 1 Diabetes develops most often in young people but can appear in adults. Type 2 Diabetes develops most often in middle aged and older adults, but can appear in young people. Diabetes is a disease derived from multiple causative factors and characterized by elevated levels of plasma glucose (hyperglycemia) in the fasting state or after administration of glucose during an oral glucose tolerance test. A decrease in β-cell mass occurs in both Type 1 and Type 2 Diabetes.

Type 1 Diabetes is an autoimmune disease that results in destruction of insulin-producing beta cells of the pancreas. Lack of insulin causes an increase of fasting blood glucose (around 70-120 mg/dL in nondiabetic people) that begins to appear in the urine above the renal threshold (about 190-200 mg/dl in most people). Type 1 Diabetes can be diagnosed using a variety of diagnostic tests that include, but are not limited to, the following: (1) glycated hemoglobin (A1C) test, (2) random blood glucose test and/or (3) fasting blood glucose test.

The glycated hemoglobin (A1C) test is a blood test that reflects the average blood glucose level of a subject over the preceding two to three months. The test measures the percentage of blood glucose attached to hemoglobin, which correlates with blood glucose levels (e.g., the higher the blood glucose levels, the more hemoglobin is glycosylated). An A1C level of 6.5 percent or higher on two separate tests is indicative of Diabetes. A result between 6 and 6.5 percent is considered prediabetic, which indicates a high risk of developing Diabetes.

The Random Blood Glucose Test comprises obtaining a blood sample at a random time point from a subject suspected of having Diabetes. Blood glucose values can be expressed in milligrams per deciliter (mg/dL) or millimoles per liter (mmol/L). A random blood glucose level of 200 mg/dL (11.1 mmol/L) or higher indicates the subject likely has Diabetes, especially when coupled with any of the signs and symptoms of Diabetes, such as frequent urination and extreme thirst.

For the fasting blood glucose test, a blood sample is obtained after an overnight fast. A fasting blood glucose level less than 100 mg/dL (5.6 mmol/L) is considered normal. A fasting blood glucose level from 100 to 125 mg/dL (5.6 to 6.9 mmol/L) is considered prediabetic, while a level of 126 mg/dL (7 mmol/L) or higher on two separate tests is indicative of Diabetes.

Type 1 Diabetes can also be distinguished from type 2 Diabetes using a C-peptide assay, which is a measure of endogenous insulin production. The presence of anti-islet antibodies (to Glutamic Acid Decarboxylase, Insulinoma Associated Peptide-2 or insulin), or lack of insulin resistance, determined by a glucose tolerance test, is also indicative of type 1, as many type 2 diabetics continue to produce insulin internally, and all have some degree of insulin resistance.

Testing for GAD 65 antibodies has been proposed as an improved test for differentiating between type 1 and type 2 Diabetes as it appears that the immune system is involved in Type 1 Diabetes etiology.

The non-obese diabetic (NOD) mouse provides an animal model for the spontaneous development of Type 1 Diabetes. NOD mice develop insulitis as a result of leukocyte infiltration into the pancreatic islet, which in turn leads to the destruction of pancreatic islets and a Type 1 diabetic phenotype (Makino S, et al., (1980) Jikken Dobutsu 29 (1): 1-13; Kikutani H, and Makino S (1992) Adv. Immunol. 51: 285-322).

In some embodiments, the method further comprises selecting a subject having Type 1 Diabetes. Such a subject can be one who has been previously diagnosed with or identified as suffering from or having Type 1 Diabetes, one or more complications related to Type 1 Diabetes, or a pre-diabetic condition, and optionally, but need not have already undergone treatment for the Type 1 Diabetes, the one or more complications related to Type 1 Diabetes, or the pre-diabetic condition. A subject can also be one who is not suffering from Type 1 Diabetes or a pre-diabetic condition. A subject can also be one who has been diagnosed with or identified as suffering from Type 1 Diabetes, one or more complications related to Type 1 Diabetes, or a pre-diabetic condition, but who show improvements in known Type 1 Diabetes risk factors as a result of receiving one or more treatments for Type 1 Diabetes, one or more complications related to Type 1 Diabetes, or the pre-diabetic condition. Alternatively, a subject can also be one who has not been previously diagnosed as having Type 1 Diabetes, one or more complications related to Type 1 Diabetes, or a pre-diabetic condition. For example, a subject can be one who exhibits one or more risk factors for Type 1 Diabetes, complications related to Type 1 Diabetes, or a pre-diabetic condition, or a subject who does not exhibit Type 1 Diabetes risk factors, or a subject who is asymptomatic for Type 1 Diabetes, one or more Type 1 Diabetes-related complications, or a pre-diabetic condition. A subject can also be one who is suffering from or at risk of developing Type 1 Diabetes or a pre-diabetic condition. A subject can also be one who has been diagnosed with or identified as having one or more complications related to Type 1 Diabetes or a pre-diabetic condition as defined herein, or alternatively, a subject can be one who has not been previously diagnosed with or identified as having one or more complications related to Type 1 Diabetes or a pre-diabetic condition.

In the context of type 1 Diabetes, “treating” or “treatment” refers to at least partial inhibition, delay or prevention of the progression of type 1 Diabetes, pre-diabetic conditions, and complications associated with type 1 Diabetes or pre-diabetic conditions; inhibition, delay or prevention of the recurrence of type 2 Diabetes, pre-diabetic conditions, or complications associated with type 1 Diabetes or pre-diabetic conditions; or the prevention of the onset or development of type 1 Diabetes, pre-diabetic conditions, or complications associated with type 1 Diabetes or pre-diabetic conditions (chemoprevention) in a subject.

In the context of Type 1 Diabetes, “therapeutically effective amount” refers to an amount of a therapeutic agent administered to a subject that is sufficient to produce a statistically significant, measurable change in at least one symptom of Type 1 Diabetes, such as glycosylated hemoglobin level, fasting blood glucose level, and hypoinsulinemia. Efficacy of treatment with a peptide can be assessed by measuring changes in blood glucose and/or insulin levels or as described below.

The efficacy of a given treatment for Type 1 Diabetes can be determined by the skilled clinician. However, a treatment is considered “effective treatment,” as the term is used herein, if any one or all of the signs or symptoms of Type 1 Diabetes, for example, hyperglycemia are altered in a beneficial manner, other clinically accepted symptoms or markers of disease are improved, or even ameliorated, e.g., by at least 10% following treatment with a peptide as described herein. Efficacy can also be measured by a failure of an individual to worsen as assessed by hospitalization or need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the loss of beta cells; or (2) relieving the disease, e.g., causing regression of symptoms, increasing pancreatic beta cell mass; and (3) preventing, slowing down development or reducing the likelihood of the development of a complication of Type 1 Diabetes, e.g., diabetic retinopathy.

An effective amount for the treatment of Type 1 Diabetes means that amount which, when administered to a subject in need thereof, is sufficient to result in effective treatment as that term is defined herein. Efficacy of a peptide can be determined by assessing physical indicators of Type 1 Diabetes, for example hyperglycemia, normoglycemia, ketone bodies, hypoinsulinemia, etc.

In one embodiment, treatment with a silk-based drug delivery composition described herein is considered effective if there is an increase of at least 10% in the level of adiponectin or a decrease of at least 10% in interleukin-6 (IL-6) measured in the subject following onset of treatment. In another embodiment, treatment with a silk-based drug delivery composition described herein is considered effective if there is an increase of at least 10% in the level of resistin measured following onset of treatment. A peptide described herein can be administered with additional therapeutic agents used to treat Type 1 Diabetes.

As used herein, the term “selecting a subject having Type 1 Diabetes” refers to the diagnosis of a subject with Type 1 Diabetes prior to the onset of treatment of the subject with a peptide as described herein. Type 1 Diabetes can be diagnosed using a glycosylated hemoglobin (A1C) test, a random blood glucose teat and/or a fasting blood glucose test. Parameters for diagnosis of Diabetes are known in the art and available to skilled artisan without much effort.

As used herein, the term “increased pancreatic beta cell mass” refers to an increase in the pancreatic beta cell mass in a subject being treated with a peptide described herein of at least 5% compared to the pancreatic beta cell mass in the subject prior to the onset of treatment. Preferably the increase in beta cell mass is at least 10%, at least 20%, at least 30%, at least 40%, at least 50%, at least 60%, at least 70%, at least 80%, at least 90%, at least 1-fold, at least 2-fold, at least 5-fold, at least 10-fold, at least 100 fold or more in a subject treated with a peptide described herein compared to the pancreatic beta cell mass in the subject prior to treatment onset. In one embodiment, beta cell mass is determined by obtaining a blood sample from a treated subject and measuring insulin levels. An increase in insulin production from the subject's beta cells is an indirect measure of the number of beta cells in the treated subject.

As used herein, the term “increase in the level of adiponectin” refers to an increase in the level of adiponectin (as measured by e.g., an adiponectin ELISA assay) of at least 10% in a subject being treated with a peptidedescribed herein compared to the level of adiponectin in the subject prior to treatment onset or compared to the level of adiponectin an untreated subject. Preferably, the increase in level of adiponectin is at least 20%, at least 30%, at least 40%, at least 50%, at least 60%, at least 70%, at least 80%, at least 90%, at least 1-fold, at least 2-fold, at least 5-fold, at least 10-fold, at least 100 fold or more in a subject treated with a peptide described herein compared to the level of adiponectin in the subject prior to treatment onset.

As used herein, the term “decrease in the level of interleukin-6 (IL-6)” refers to a decrease in the level of IL-6 (as measured by e.g., an IL-6 ELISA assay) of at least 10% in a subject being treated with a peptide described herein compared to the level of IL-6 in the subject prior to treatment onset. Preferably, the decrease in interleukin-6 is at least 20%, at least 30%, at least 40%, at least 50%, at least 60%, at least 70%, at least 80%, at least 90%, or more compared to the level of interleukin-6 in the subject prior to treatment with a peptide as described herein.

As used herein, the term “HBAlc” refers to glycosylated hemoglobin or glycated hemoglobin, and is an indicator of blood glucose levels over a period of time (e.g., 2-3 months). The level of HBAlc is “reduced” if there is a decrease of at least 20%, at least 30%, at least 40%, at least 50%, at least 60%, at least 70%, at least 80%, at least 90%, at least 95%, or more upon treatment with a peptide described herein compared to the level of HBAlc prior to the onset of treatment in the subject. Similarly, ketone bodies are “reduced” if there is a decrease of at least 20%, at least 30%, at least 40%, at least 50%, at least 60%, at least 70%, at least 80%, at least 90%, at least 95%, or more upon treatment with a peptide described herein.

As used herein, the term “delaying the onset of Type 1 Diabetes” in a subject refers to a delay of onset of at least one symptom of Type 1 Diabetes (e.g., hyperglycemia and/or hypoinsulinemia) of at least one week, at least 2 weeks, at least 1 month, at least 2 months, at least 6 months, at least 1 year, at least 2 years, at least 5 years, at least 10 years, at least 20 years, at least 30 years, at least 40 years or more, and can include the entire lifespan of the subject.

Type 2 Diabetes results from a combination of insulin resistance and impaired insulin secretion but ultimately many people with Type 2 Diabetes show markedly reduced pancreatic β-cell mass and function which, in turn, causes Type 2 diabetic persons to have a “relative” deficiency of insulin because pancreatic β-cells are producing some insulin, but the insulin is either too little or isn't working properly to adequately allow glucose into cells to produce energy. Recent autopsy studies have shown clear evidence of ongoing β-cell death (apoptosis) in people with Type 2 Diabetes. Therefore, therapeutic approaches to provide more β-cells could provide a significant treatment for reversing or curing Type 2 Diabetes.

Uncontrolled Type 2 Diabetes leads to excess glucose in the blood, resulting in hyperglycemia, or high blood sugar. A person with Type 2 Diabetes experiences fatigue, increased thirst, frequent urination, dry, itchy skin, blurred vision, slow healing cuts or sores, more infections than usual, numbness and tingling in feet. Without treatment, a person with Type 2 Diabetes will become dehydrated and develop a dangerously low blood volume. If Type 2 Diabetes remains uncontrolled for a long period of time, more serious symptoms may result, including severe hyperglycemia (blood sugar over 600 mg) lethargy, confusion, shock, and ultimately “hyperosmolar hyperglycemic non-ketotic coma” Persistent or uncontrolled hyperglycemia is associated with increased and premature morbidity and mortality. As such, therapeutic control of glucose homeostasis, lipid metabolism, obesity, and hypertension are critically important in the clinical management and treatment of Diabetes mellitus.

The silk-based drug delivery compositions and methods described herein are useful for treating type 2 Diabetes Mellitus or a pre-diabetic condition in a subject or preventing type 2 Diabetes or pre-diabetic conditions in a subject. Skilled artisan is well aware that type 2 Diabetes Mellitus is also known as non-insulin dependent Diabetes mellitus. In one aspect the invention provides a method of treating type 2 Diabetes in a subject comprising administering a silk-based drug delivery composition described herein.

In some embodiments of this aspect, the method further comprises selecting a subject having Type 2 Diabetes or a pre-diabetic condition. Such a subject can be one who has been previously diagnosed with or identified as suffering from or having Type 2 Diabetes, one or more complications related to Type 2 Diabetes, or a pre-diabetic condition, and optionally, but need not have already undergone treatment for the Type 2 Diabetes, the one or more complications related to Diabetes, or the pre-diabetic condition. A subject can also be one who is not suffering from Type 2 Diabetes or a pre-diabetic condition. Such subject may otherwise be at risk of diabetes, such as a carrier of one or more predisposing mutations that increase the likelihood of developing diabetes. A subject can also be one who has been diagnosed with or identified as suffering from Type 2 Diabetes, one or more complications related to Type 2 Diabetes, or a pre-diabetic condition, but who show improvements in known Type 2 Diabetes risk factors as a result of receiving one or more treatments for Type 2 Diabetes, one or more complications related to Type 2 Diabetes, or the pre-diabetic condition. Alternatively, a subject can also be one who has not been previously diagnosed as having Type 2 Diabetes, one or more complications related to Type 2 Diabetes, or a pre-diabetic condition. For example, a subject can be one who exhibits one or more risk factors for Type 2 Diabetes, complications related to Type 2 Diabetes, or a pre-diabetic condition, or a subject who does not exhibit Type 2 Diabetes risk factors, or a subject who is asymptomatic for Type 2 Diabetes, one or more Type 2 Diabetes-related complications, or a pre-diabetic condition. A subject can also be one who is suffering from or at risk of developing Type 2 Diabetes or a pre-diabetic condition. A subject can also be one who has been diagnosed with or identified as having one or more complications related to Type 2 Diabetes or a pre-diabetic condition as defined herein, or alternatively, a subject can be one who has not been previously diagnosed with or identified as having one or more complications related to Type 2 Diabetes or a pre-diabetic condition.

“Complications related to type 2 Diabetes” or “complications related to a pre-diabetic condition” can include, without limitation, diabetic retinopathy, diabetic nephropathy, blindness, memory loss, renal failure, cardiovascular disease (including coronary artery disease, peripheral artery disease, cerebrovascular disease, atherosclerosis, and hypertension), neuropathy, autonomic dysfunction, hyperglycemic hyperosmolar coma, or combinations thereof.

In the context of type 2 Diabetes, “treating” or “treatment” refers to some inhibition, delay or prevention of the progression of type 2 Diabetes, pre-diabetic conditions, and complications associated with type 2 Diabetes or pre-diabetic conditions; inhibition, delay or prevention of the recurrence of type 2 Diabetes, pre-diabetic conditions, or complications associated with type 2 Diabetes or pre-diabetic conditions; or the prevention of the onset or development of type 2 Diabetes, pre-diabetic conditions, or complications associated with type 2 Diabetes or pre-diabetic conditions (chemoprevention) in a subject.

In the context of type 2 Diabetes, “therapeutically-effective amount” can also refer to an amount that is effective to induce an inhibition of kinase activity from one or more kinases implicated in type 2 Diabetes Mellitus or pre-diabetic conditions as defined herein. The inhibitory amount may be determined directly by measuring the inhibition of kinase activity, or, for example, where the desired effect is an effect on an activity downstream of a particular kinase activity in a pathway that includes one or more kinases involved in Diabetes or a pre-diabetic condition, the inhibition may be measured by measuring a downstream effect. Thus, the inhibition of kinase activity will depend in part on the nature of the inhibited pathway or process that involves kinase activity, and on the effects that inhibition of kinase activity has in a given biological context.

Potentiation of insulin signaling in vivo, which can result from administration of the pharmaceutical compositions described herein, can be monitored as a clinical endpoint. In principle, a way to look at insulin potentiation in a patient is to perform an oral glucose tolerance test. After fasting, glucose is given to a patient and the rate of the disappearance of glucose from blood circulation (namely glucose uptake by cells) is measured by assays well known in the art. Slow rate (as compared to healthy subject) of glucose clearance will indicate insulin resistance. The administration of one or more peptides of the inhibitors of the invention, to an insulin-resistant subject can increase the rate of glucose uptake as compared to a non-treated subject. The silk-based drug delivery composition can be administered to an insulin resistant subject for a longer period of time, and the levels of insulin, glucose, and leptin in blood circulation (which are usually high) can be determined. Decrease in glucose levels will indicate that the silk-based drug delivery composition potentiated insulin action. A decrease in insulin and leptin levels alone may not necessarily indicate potentiation of insulin action, but rather will indicate improvement of the disease condition by other mechanisms.

The silk-based drug delivery compositions described here can be used to therapeutically treat Diabetes or a pre-diabetic condition in a patient with type 2 Diabetes or a pre-diabetic condition as defined herein. A therapeutically effective amount of the therapeutic agent, e.g., GLP-1 receptor agonist, can be administered to the patient, and clinical markers, for example blood sugar level and/or IRS-1 phosphorylation, can be monitored.

Exemplary embodiments of the invention can be also described by any one of the following numbered paragraphs:
1. A sustained drug delivery composition, the composition comprising
   (i) a silk matrix comprising silk fibroin; and
   (ii) a glucagon-like peptide (GLP-1) receptor agonist;
   wherein the agonist is dispersed or encapsulated in the silk matrix.
2. The composition of paragraph 1, wherein the silk matrix is selected from the group consisting of hydrogel, microparticle, nanoparticle, fiber, film, lyophilized powder, lyophilized gel, reservoir implant, homogenous implant, gel-like or gel particle, and any combinations thereof
3. The composition of any of paragraphs 1-2, wherein the composition comprises from about 0.1% to about 50% (w/v or w/w) of the silk fibroin.
4. The composition of any of paragraphs 1-3, wherein the composition comprises about 1% to about 30% (w/v or w/w) of the silk fibroin.
5. The composition of any of paragraphs 1-4, wherein the GLP-1 receptor agonist is selected from the group consisting of metformin (Glucophage, Glumetza), pioglitazone (Actos), glyburide (DiaBeta, Glynase), glipizide (Glucotrol), glimepiride (Amaryl), repaglinide (Prandin), nateglinide (Starlix), sitagliptin (Januvia), saxagliptin (Onglyza), exenatide (Byetta), liraglutide (Victoza), insulin lispro (Humalog), insulin aspart (NovoLog), insulin glargine (Lantus), insulin detemir (Levemir), and any combination thereof
6. The composition of any of paragraphs 1-5, wherein the GLP-1 receptor agonist is exenatide or liraglutide.
7. The composition of any of paragraphs 1-6, wherein the composition comprises from about 0.01% to about 95%(w/v or w/w) of the GLP-1 receptor agonist.
8. The composition of any of paragraphs 1-7, wherein the composition comprises from about 0.01% to about 5%(w/v or w/w) of the GLP-1 receptor agonist.
9. The composition of paragraph 8, wherein the composition comprises about 0.06% to about 0.42% (w/v or w/w) of the GLP-1 receptor agonist.
10. The composition of any of paragraphs 1-9, wherein the silk matrix further comprises a biocompatible polymer.
11. The composition of paragraph 10, wherein the biocompatible polymer is dispersed or encapsulated in the silk matrix.
12. The composition of paragraph 10 or 11, wherein the biocompatible polymer is selected from the group consisting of a poly-lactic acid (PLA), poly-glycolic acid (PGA), poly-lactide-co-glycolide (PLGA), polyesters, poly(ortho ester), poly(phosphazine), poly(phosphate ester), polycaprolactone, gelatin, collagen, poly(ethylene glycol) (PEG), polyethylene oxide (PEO), triblock copolymers, polylysine and any derivatives thereof
13. The composition of paragraph 12, wherein the biocompatible polymer is PEG of molecular weight about 10,000 or PEO of molecular weight about 100,000.
14. The composition of any of paragraphs 10-13, wherein the composition comprises from about 0.1% to about 25% (w/v) of the biocompatible polymer.
15. The composition of paragraph 14, wherein the composition comprises from about 0.25% to about 5% (w/v or w/w) of the biocompatible polymer
16. The composition of any of paragraphs 1-15, wherein the composition further comprises albumin.
17. The composition of paragraph 16, wherein the albumin is dispersed or encapsulated in the silk matrix.
18. The composition of paragraph 16 or 17, wherein the albumin is bovine serum albumin.
19. The composition of paragraph 16 or 17, wherein the albumin is human serum albumin.
20. The composition of any of paragraphs 16-19, wherein amount of albumin in the composition is from about 0.5% to about 25% (w/v or w/w).
21. The composition of paragraph 20, wherein amount of albumin in the composition is about 5% (w/v or w/w).
22. The composition of any of paragraphs 1-20, wherein the composition is injectable.
23. The composition of any of paragraphs 1-22, wherein the composition comprises:
   (i) about 2%, about 4%, about 8%, about 10%, or about 16% (w/v) of silk fibroin;
   (ii) about 0.06% (w/v), about 0.12% (w/v), or about 0.42% (w/v) of the GLP-1 receptor agonist, wherein the GLP-1 receptor agonist is exenatide or liraglutide; and
   (iii) optionally about 1% (w/v) of PEO (MW 100,000) or 5% (w/v) of PEG (MW10,000).
24. The composition of any of paragraphs 1-22, wherein the composition comprise:
   (i) about 2%, about 4%, about 8%, about 10%, or about 16% (w/v) of silk fibroin;
   (ii) about 0.06% (w/v), about 0.12% (w/v), or about 0.42% (w/v) of the GLP-1 receptor agonist, wherein the GLP-1 receptor agonist is exenatide or liraglutide; and
   (iii) optionally about 5% (w/v) of albumin.
25. The composition of any of paragraphs 1-24, wherein the composition provides sustain release of the GLP-1 receptor agonist over a period of at least about a week.
26. The composition of any of paragraphs 1-25, wherein the GLP-1 receptor agonist is released from the silk matrix at a rate of from about 5 μg/day to about 60 μg/day.
27. The composition of paragraph 26, wherein the GLP-1 receptor agonist is released from the silk matrix at a rate of about 10 μg/day.
28. The composition of any of paragraphs 1-27, wherein the GLP-1 receptor agonist has duration of therapeutic effect which is at least one day longer relative to duration of therapeutic effect in the absence of the silk matrix.
29. A pharmaceutical composition comprising a sustained delivery composition of any of paragraphs 1-28 and a pharmaceutically acceptable carrier.
30. A method for treating diabetes or pre-diabetic condition in a subject, the method comprising administering to a subject in need thereof a composition of any of paragraphs 1-29.
31. The method of paragraph 30, wherein administration frequency of the composition is less than when the same amount of GLP-1 receptor agonist is administered in the absence of the silk matrix.
32. The method of paragraph 31, wherein the administration frequency is reduced by a factor of ½ relative to when the GLP-1 receptor agonist is administered in the absence of the silk matrix.
33. The method of any of paragraphs 30-32, wherein said administration is no more than once a month, no more than once every two week, no more than once every three weeks, no more than once a month, no more than once every two months, no more than once every four months or no more once every six months.
34. A drug delivery device comprising the composition of any of paragraphs 1-28.
35. The drug delivery device of paragraph 34, wherein the drug delivery device is a syringe with an injection needle.
36. The drug delivery device of paragraph 35, wherein the device is an implant.
37. A kit comprising a composition of any of paragraphs 1-28, or a drug delivery device of any of paragraphs of 34-36.
38. The kit of paragraph 37, further comprising at least a syringe and an injection needle.
39. The kit of any of paragraphs 37-38, further comprising an anesthetic.
40. The kit of any of paragraphs 37-39, further comprising an antiseptic agent.
41. The kit of any of paragraphs 37-40, further comprising instruction for use.
42. A method for preparing a sustained delivery composition of any of paragraphs 1-28, the method comprising:
   (i) providing a silk solution comprising silk fibroin and a glucagon-like peptide (GLP-1) receptor agonist; and
   (ii) inducing gelation in the silk solution to form a silk hydrogel, wherein the GLP-1 receptor agonist becomes dispersed or encapsulated within the silk hydrogel.
43. The method of paragraph 42, wherein said inducing gelation is by applying shear stress, applying sonication or ultrasonication, modulating the pH of the silk solution, or any combination thereof.

### Some Selected Definitions

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are useful to an embodiment, yet open to the inclusion of unspecified elements, whether useful or not.

The singular terms “a,” “an,” and “the” include plural referents unless context clearly indicates otherwise. Similarly, the word “or” is intended to include “and” unless the context clearly indicates otherwise.

Other than in the operating examples, or where otherwise indicated, all numbers expressing quantities of ingredients or reaction conditions used herein should be understood as modified in all instances by the term “about.” The term “about” when used in connection with percentages may mean±5% of the value being referred to. For example, about 100 means from 95 to 105.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means at least two standard deviation (2SD) away from a reference level. The term refers to statistical evidence that there is a difference. It is defined as the probability of making a decision to reject the null hypothesis when the null hypothesis is actually true.

As used interchangeably herein, the terms “essentially” and “substantially” mean a proportion of at least about 60%, or preferably at least about 70% or at least about 80%, or at least about 90%, at least about 95%, at least about 97% or at least about 99% or more, or any integer between 70% and 100%. In some embodiments, the terms “essentially” and “substantially” mean a proportion of at least about 90%, at least about 95%, at least about 98%, at least about 99% or more, or any integer between 90% and 100%. In some embodiments, the terms “essentially” and “substantially” do not include 100%. In some embodiments, the terms “essentially” and “substantially” can include 100%.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

The disclosure is further illustrated by the following examples which should not be construed as limiting. The examples are illustrative only, and are not intended to limit, in any manner, any of the aspects described herein. The following examples do not in any way limit the invention.

### EXAMPLES

There are a wide variety of drugs used to control blood glucose levels that are administered on a daily basis including metformin (Glucophage, Glumetza), pioglitazone (Actos), glyburide (DiaBeta, Glynase), glipizide (Glucotrol), glimepiride (Amaryl), repaglinide (Prandin), nateglinide (Starlix), sitagliptin (Januvia), and saxagliptin (Onglyza), which are all prescribed as oral tablets. Other daily options include exenatide (Byetta) and liraglutide (Victoza), which are prescribed as daily subcutaneous injections. In addition, insulin therapy, ranging from rapid-acting to long-acting insulin as well as insulin pumps, may be used with drugs including insulin lispro (Humalog), insulin aspart (NovoLog), insulin glargine (Lantus), and insulin detemir (Levemir) among the most commonly prescribed. These drugs are administered before/after meals or once daily as a subcutaneous injection (long-acting). One longer-term formulation is Bydureon, a long-acting release version of exenatide using PLGA microspheres developed by Amylin Pharmaceuticals, Eli Lilly & Co., and Alkermes. It recently received FDA approval and is administered as a once weekly subcutaneous injection.

### Materials and Methods

Preparation of Sterile, Low-Endotoxin Aqueous Silk Fibroin Solution:

Aqueous silk fibroin solutions (6-8% (w/v)) were prepared sterilely from degummed silk fibers from Soho Biomaterials Co. (Suzhou, China) using aseptic techniques. Briefly, the sterile silk fibers were dissolved in 9.3 M lithium bromide and dialyzed against deionized water for 48 hours. Resulting silk solutions were concentrated, if necessary, by dialyzing against poly(ethylene glycol) (PEG) to produce 20-35% (w/v) silk fibroin solutions. All solutions were stored at 4° C. until used to make drug formulations.

Preparation of Liraglutide-Loaded Silk Hydrogel Formulations:

Liraglutide-loaded silk hydrogel formulations were prepared by mixing silk (4 to 16% (w/v)) and liraglutide (0.6% (w/v)) solutions to achieve the desired final concentrations of silk and liraglutide in the gel formulation. To induce gelation, these solutions were sonicated using a digital sonifier (Branson) before preparing the solutions for injection by drawing the solutions into 1 mL syringes using a 16-18 G needle, withdrawing air from the syringe, and replacing the needle with a 21-30 G needle suitable for injection. The syringes were incubated for 1-2 days at 37° C. before switching to 4° C. for storage (if necessary) prior to injection.

Preparation of Exenatide-Loaded Silk Hydrogel Formulations:

Exenatide-loaded silk hydrogel formulations were prepared by mixing silk (4 to 32% (w/v)) and exenatide (0.12 to 0.48% (w/v)) solutions to achieve the desired final concentrations of silk and exenatide in the gel formulation. As an example, for the 4% silk/0.06% exenatide gels, equal volumes of sterile 8% silk and exenatide (0.12%) were mixed to achieve final concentrations of 4% and 0.06%, respectively. Some formulations were prepared with different additives to modify release kinetics, including polyethylene glycol (PEG, MW 10,000, 0 to 5% (w/v)), polyethylene oxide (PEO, MW 100,000, 0 to 1% (w/v)), and bovine serum albumin (BSA, 0 to 5% (w/v)). To induce gelation, these solutions were sonicated using a digital sonifier (Branson) under aseptic conditions before preparing the solutions for injection by drawing the solutions into 1 mL syringes using a 16-18 G needle, withdrawing air from the syringe, and replacing the needle with a 21-30 G needle suitable for injection. The syringes were incubated at 37° C. until gelation before switching to 4° C. for storage (if necessary) before injection.

In Vitro Evaluation of Drug-Loaded Silk Hydrogel Formulations:

Release kinetics in vitro were determined by incubating the drug-loaded silk hydrogel formulations in phosphate buffered saline (PBS) solution and/or Sprague-Dawley rat plasma for up to 67 days. Briefly, liraglutide- or exenatide-loaded silk hydrogels were injected (100 μL/injection) or aliquot via pipette into 4 mL of PBS with 0.02% (w/v) sodium azide or Sprague-Dawley rat plasma (Innovative Research), with release medium sampled (3.6 mL/sample) and replaced at 2, 6, and 24 hour timepoints and then every 1-3 days thereafter. Samples were analyzed for liraglutide or exenatide concentrations using commercially available enzyme-linked immunosorbent assay (ELISA) kits (AB Biolabs, Ballwin, Mo.; Phoenix Pharmaceuticals, Burlingame, Calif.), following kit instructions.

Pharmacokinetic Evaluation of Exenatide-Loaded Silk Formulations:

Pharmacokinetic properties of exenatide-loaded silk hydrogels were evaluated following subcutaneous injection in Sprague-Dawley rats. Levels of exenatide in the blood plasma were evaluated over the course of the experiment according to the protocol outlined by Agilux Laboratories (Worcester, Mass.), a preclinical contract research organization and the performing laboratory for the study. The amount of exenatide in each sample was determined using an ELISA kit (AB Biolabs, Ballwin, Mo.).

### Results and Discussion

In Vitro Formulation Development of Liraglutide-Loaded Silk Hydrogel Formulations:

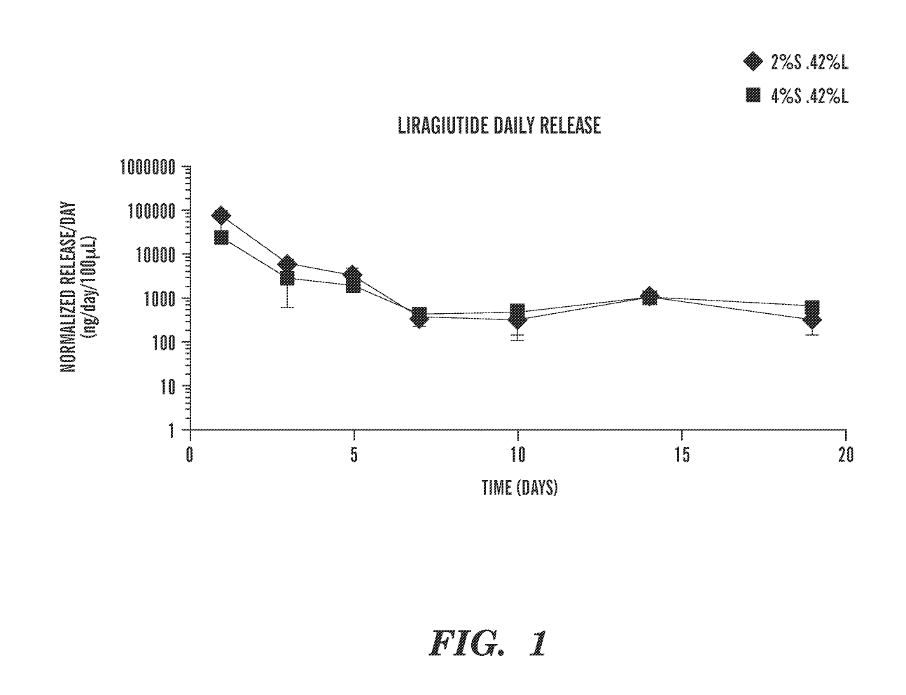

Liraglutide-loaded hydrogel formulations were prepared using different silk gel concentrations (2% and 4% (w/v)). Liraglutide was loaded in the gels at a final concentration of 0.42% (w/v). As shown in FIG. 1, the 4% silk gels had a slightly lower burst release over the first 5 days as compared to the 2% gels, with both gels demonstrating sustained release from day 7 to 19. While this initial proof-of-concept data is encouraging, future work will focus on higher loading of liraglutide (up to 10% (w/v)) and higher silk concentrations (up to 24% (w/v)).

Pharmacokinetic Evaluation of Exenatide-Loaded Silk Hydrogel Formulations:

The pharmacokinetic evaluation of exenatide-loaded silk hydrogels in Sprague-Dawley rats was conducted by Agilux Laboratories. The dosing design and sampling collection schedule for the study is provided in Tables 1 and 2, respectively.

TABLE 1
Dosing Design

| | Number | | Dose | | | | |
|---|---|---|---|---|---|---|---|
| Group | of | Test | Level | Concentration | Dose | | |
| Number | Males | Article | (mg/rat) | (mg/mL) | Volume | Vehicle | Route |
| 1 | 3 | Exenatide | 0.6 | 0.6 | 1 | 2% GEL | SC |
| | | | | | ML/RAT | FORMULATION | |
| 2 | 3 | Exenatide | 0.6 | 0.6 | 1 | 4% GEL | SC |
| | | | | | ML/RAT | FORMULATION | |
| 3 | 3 | N/A | 0 | 0 | 1 | 2% GEL | SC |
| | | | | | ML/RAT | FORMULATION | |
| 4 | 3 | N/A | 0 | 0 | 1 | 4% GEL | SC |
| | | | | | ML/RAT | FORMULATION | |
| 5 | 3 | Exenatide | 0.6 | 0.6 | 1 | AQUEOUS | SC |
| | | | | | ML/RAT | | |

TABLE 2
Sample Collection

| Group Number | Serial Blood Collection Time |
|---|---|
| All | Pre-Dose, 2, 6, 24 hours (Day 1) and Day 2, 3, 7, 10, |
| | 14, and 16 |
| Anticoagulant | K₂EDTA |
| Volume/Timepoint | ~200-300 μL |

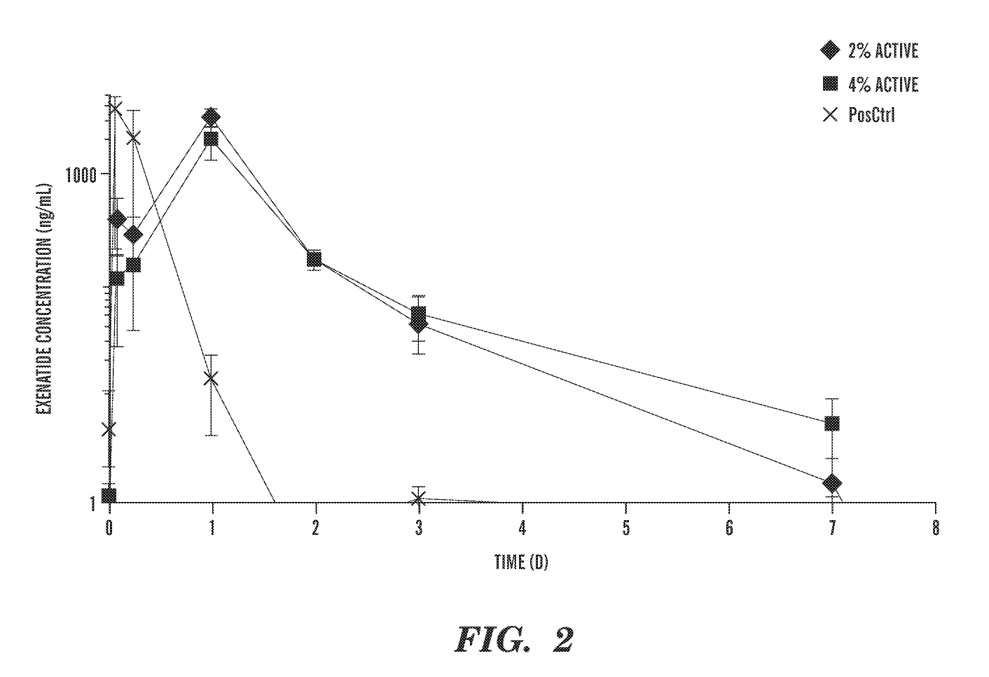

Male Sprague-Dawley rats (300 g+) were dosed by single subcutaneous injection of 1 mL/animal. Over the course of the study, the animals were observed with special attention given to administration sites to assess test article absorption, reactivity, and healing. Serial blood samples were harvested via the tail or jugular vein according to the schedule outlined in Table 2, and processed to plasma according to the study protocol. The data was analyzed using an ELISA method and plotted in FIG. 2.

As shown in FIG. 2, the exenatide concentration level at day 7 for the 2 active groups (2% and 4% silk, 0.06% exenatide) is approximately equivalent to the exenatide concentration for the positive control group at day 1 (0.06% exenatide solution injection). This suggests that these two gel formulations can provide approximate therapeutic levels of exenatide for an extended period of time beyond that of the solution control. In the case of the positive solution control, exenatide levels were below quantification levels after day 3, further emphasizing the improvement in sustaining the delivery of exenatide using silk gel formulations.

In Vitro Formulation Development of Exenatide-Loaded Silk Hydrogel Formulations:

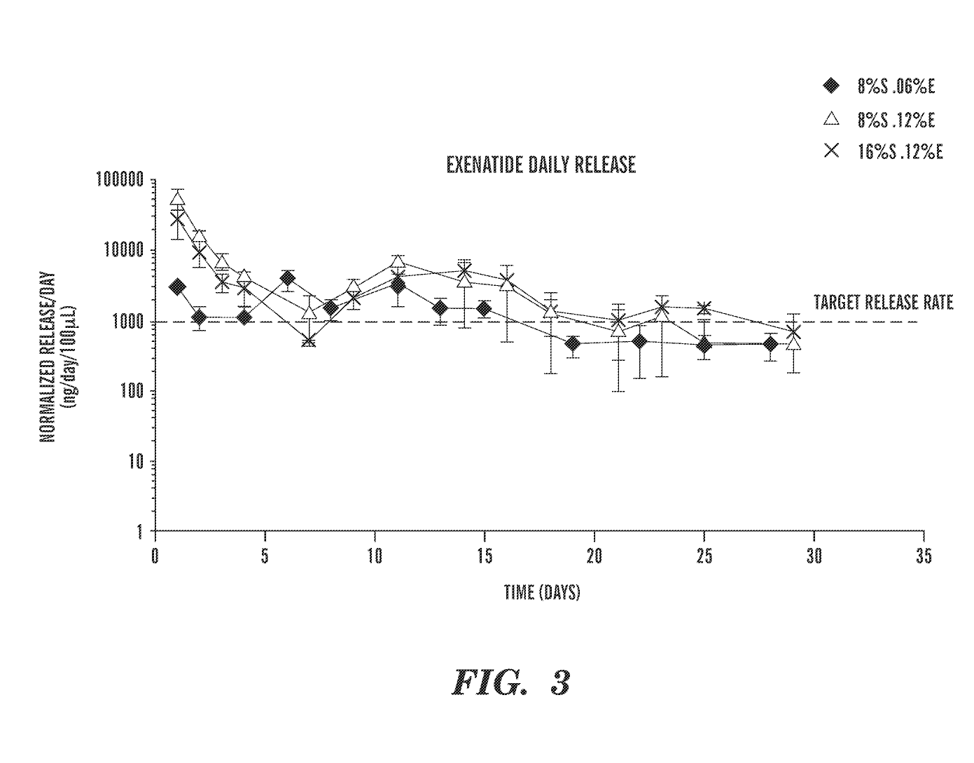

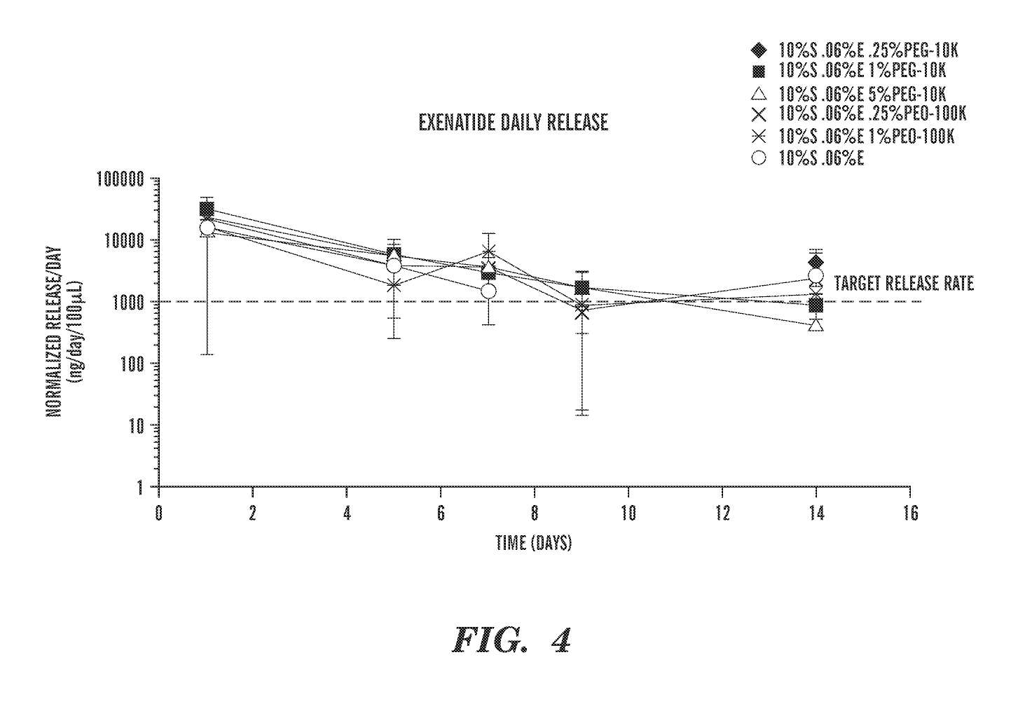

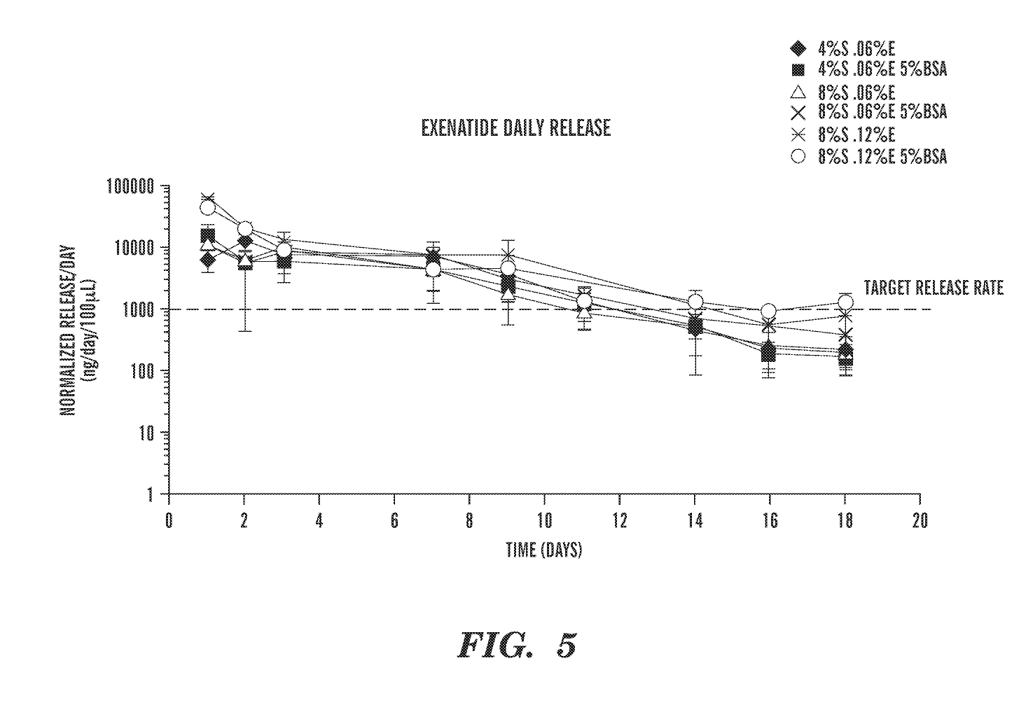

Further improvements to the gel formulations have been assessed using in vitro release studies. These silk hydrogel samples have focused on increased drug loading (up to 0.24% (w/v) exenatide), increased silk concentration (up to 16% silk), as well as different gel additives to alter the release kinetics (e.g., polyethylene glycol, polyethylene oxide, and bovine serum albumin). Formulation concentrations of polyethylene glycol (PEG, MW 10,000) ranged from 0 to 5% (w/v), concentrations of polyethylene oxide (PEO, MW 100,000) ranged from 0 to 1% (w/v), and concentrations of bovine serum albumin (BSA) ranged from 0 to 5% (w/v). Samples were collected and analyzed as described above over the course of up to 67 days, with the formulations displaying the most promising release kinetics shown in FIG. 3. Examples of formulations with and without PEG, PEO, and BSA are provided in FIGS. 4 and 5.

Based on the results of this liraglutide- and exenatide-loaded silk formulation development and the accompanying pilot rat pharmacokinetic study, the work described herein shows that it is possible to deliver GLP-1 receptor agonist therapeutics using a silk hydrogel formulation and achieve sustained release for 1-2 months or longer. With 2% and 4% silk gels, exenatide release was sustained for one week, with further improvements in vitro with higher concentration silk gels (up to 16% (w/v)) demonstrating sustained release out to one month at or near the target therapeutic range. Dosage volume can be adjusted to achieve the target range of 5-60 μg/day and plasma concentrations of 100-385 μg/mL (Fineman et al., Clinical Pharmacokinetics 50 (2011), 65). While the work reported herein focused on exenatide, other GLP-1 receptor agonist therapeutics, such as liraglutide, can also be used. Accordingly, the compositions and methods described herein can be used for sustained delivery of antibodies, peptides, small molecules, and nucleic acid based therapeutics (e.g. RNAi therapeutics) and can be used for the treatment of a wide range of diseases beyond diabetes mellitus.

As described herein compositions and method has been developed for formulating glucagon-like peptide (GLP-1) receptor agonist therapeutics for sustained release. This formulation can be used to reduce the frequency of dosing for patients currently enduring treatment using these GLP-1 receptor agonists. In embodiments of the compositions, the composition comprised different concentrations of GLP-1 receptor agonists (up to 0.42% (w/v)) loaded within different concentration silk hydrogels (up to 24% (w/v)). The gels are formed using sonication according to prior IP disclosures (see Wang et al., U.S. Ser. No. 12/601,845; PCT/US2008/065076) and loaded into syringes for injection. Exemplary compositions were made using exenatide and liraglutide as the GLP-1 receptor agonists in the hydrogels, determining release kinetics for up to 2 months in experiments in vitro as well as 1 week in vivo using a subcutaneous injection model in Sprague-Dawley rats. One formulation for in vivo was 0.06% exenatide loaded in a 4% silk hydrogel. These formulations demonstrated release concentrations at day 7 that are equivalent to those achieved with the positive control (0.06% exenatide solution injection) at the 1 day time point. In in vitro experiments, further refinement of the formulations has shown that increased silk concentrations in the hydrogels (up to 24% (w/v)) can extend the release of exenatide up to 2 months, indicating that the patient injections can be repeated less frequently (e.g., one injection every 2 months or longer) than the current standard of care.

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

What is claimed is:

1. A sustained drug delivery composition, the composition comprising
(i) a silk matrix comprising silk fibroin; and
(ii) a glucagon-like peptide (GLP-1) receptor agonist;
wherein the agonist is dispersed or encapsulated in the silk matrix.

2. The composition of claim 1, wherein the silk matrix is selected from the group consisting of hydrogel, microparticle, nanoparticle, fiber, film, lyophilized powder, lyophilized gel, reservoir implant, homogenous implant, gel-like or gel particle, and any combinations thereof.

3. The composition of claim 1, wherein the composition comprises from about 0.1% to about 50% (w/v or w/w) of the silk fibroin.

4. The composition of claim 3, wherein the composition comprises about 1% to about 30% (w/v or w/w) of the silk fibroin.

5. The composition of claim 1, wherein the GLP-1 receptor agonist is selected from the group consisting of metformin (Glucophage, Glumetza), pioglitazone (Actos), glyburide (DiaBeta, Glynase), glipizide (Glucotrol), glimepiride (Amaryl), repaglinide (Prandin), nateglinide (Starlix), sitagliptin (Januvia), saxagliptin (Onglyza), exenatide (Byetta), liraglutide (Victoza), insulin lispro (Humalog), insulin aspart (NovoLog), insulin glargine (Lantus), insulin detemir (Levemir), and any combination thereof.

6. The composition of claim 5, wherein the GLP-1 receptor agonist is exenatide or liraglutide.

7. The composition of claim 1, wherein the composition comprises from about 0.01% to about 95%(w/v or w/w) of the GLP-1 receptor agonist.

8. The composition of claim 7, wherein the composition comprises from about 0.01% to about 5%(w/v or w/w) of the GLP-1 receptor agonist.

9. The composition of claim 8, wherein the composition comprises about 0.06% to about 0.42% (w/v or w/w) of the GLP-1 receptor agonist.

10. The composition of claim 1, wherein the silk matrix further comprises a biocompatible polymer.

11. The composition of claim 10, wherein the biocompatible polymer is dispersed or encapsulated in the silk matrix.

12. The composition of claim 10, wherein the biocompatible polymer is selected from the group consisting of a poly-lactic acid (PLA), poly-glycolic acid (PGA), poly-lactide-co-glycolide (PLGA), polyesters, poly(ortho ester), poly(phosphazine), poly(phosphate ester), polycaprolactone, gelatin, collagen, poly(ethylene glycol) (PEG), polyethylene oxide (PEO), triblock copolymers, polylysine and any derivatives thereof.

13. The composition of claim 12, wherein the biocompatible polymer is PEG of molecular weight about 10,000 or PEO of molecular weight about 100,000.

14. The composition of claim 10, wherein the composition comprises from about 0.1% to about 25% (w/v) of the biocompatible polymer.

15. The composition of claim 14, wherein the composition comprises from about 0.25% to about 5% (w/v or w/w) of the biocompatible polymer.

16. The composition of claim 1, wherein the composition further comprises albumin.

17. The composition of claim 16, wherein the albumin is dispersed or encapsulated in the silk matrix.

18. The composition of claim 16, wherein the albumin is bovine serum albumin.

19. The composition of claim 16, wherein the albumin is human serum albumin.

20. The composition of any of claim 16, wherein amount of albumin in the composition is from about 0.5% to about 25% (w/v or w/w).

21. The composition of claim 20, wherein amount of albumin in the composition is about 5% (w/v or w/w).

22. The composition of claim 1, wherein the composition is injectable.

23. The composition of claim 1, wherein the composition comprises:
(i) about 2%, about 4%, about 8%, about 10%, or about 16% (w/v) of silk fibroin;
(ii) about 0.06% (w/v), about 0.12% (w/v), or about 0.42% (w/v) of the GLP-1 receptor agonist, wherein the GLP-1 receptor agonist is exenatide or liraglutide; and
(iii) optionally about 1% (w/v) of PEO (MW 100,000) or 5% (w/v) of PEG (MW10,000).

24. The composition of claim 1, wherein the composition comprise:
(i) about 2%, about 4%, about 8%, about 10%, or about 16% (w/v) of silk fibroin;
(ii) about 0.06% (w/v), about 0.12% (w/v), or about 0.42% (w/v) of the GLP-1 receptor agonist, wherein the GLP-1 receptor agonist is exenatide or liraglutide; and
(iii) optionally about 5% (w/v) of albumin.

25. The composition of claim 1, wherein the composition provides sustain release of the GLP-1 receptor agonist over a period of at least about a week.

26. The composition of claim 1, wherein the GLP-1 receptor agonist is released from the silk matrix at a rate of from about 5 μg/day to about 60 μg/day.

27. The composition of claim 26, wherein the GLP-1 receptor agonist is released from the silk matrix at a rate of about 10 μg/day.

28. The composition of claim 1, wherein the GLP-1 receptor agonist has duration of therapeutic effect which is at least one day longer relative to duration of therapeutic effect in the absence of the silk matrix.

29. A pharmaceutical composition comprising a sustained delivery composition of claim 1 and a pharmaceutically acceptable carrier.

30. A method for treating diabetes or pre-diabetic condition in a subject, the method comprising administering to a subject in need thereof a composition of claim 1.

31. The method of claim 30, wherein administration frequency of the composition is less than when the same amount of GLP-1 receptor agonist is administered in the absence of the silk matrix.

32. The method of claim 31, wherein the administration frequency is reduced by a factor of ½ relative to when the GLP-1 receptor agonist is administered in the absence of the silk matrix.

33. The method of claim 30, wherein said administration is no more than once a month, no more than once every two week, no more than once every three weeks, no more than once a month, no more than once every two months, no more than once every four months or no more once every six months.

34. A drug delivery device comprising the composition of claim 1.

35. The drug delivery device of claim 34, wherein the drug delivery device is a syringe with an injection needle.

36. The drug delivery device of claim 35, wherein the device is an implant.

37. A kit comprising a composition of claim 1, or a drug delivery device of any of claim of 34.

38. The kit of claim 37, further comprising at least a syringe and an injection needle.

39. The kit of claim 37, further comprising an anesthetic.

40. The kit of claim 37, further comprising an antiseptic agent.

41. The kit of claim 37, further comprising instruction for use.

42. A method for preparing a sustained delivery composition of claim 1, the method comprising:
(i) providing a silk solution comprising silk fibroin and a glucagon-like peptide (GLP-1) receptor agonist; and
(ii) inducing gelation in the silk solution to form a silk hydrogel,
wherein the GLP-1 receptor agonist becomes dispersed or encapsulated within the silk hydrogel.

43. The method of claim 42, wherein said inducing gelation is by applying shear stress, applying sonication or ultrasonication, modulating the pH of the silk solution, or any combination thereof.
